(19) United States
(12) Patent Application Publication
(10) Pub. No.: US 2008/0262070 A1
(43) Pub. Date: Oct. 23, 2008

(54) Pharmaceutically Active Tetrahydro-Pyrrolizinone Compounds

(76) Inventor: Karl Baumann, Wien (AT)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/089,254

(22) PCT Filed: Oct. 4, 2006
(86) PCT No.: PCT/EP06/09598

§ 371 Date: Apr. 4, 2008

(30) Foreign Application Priority Data
Oct. 6, 2005 (GB) 0520376.5
Oct. 6, 2005 (GB) 0520377.3
Oct. 6, 2005 (GB) 0520379.9

(51) Int. Cl. A61K 31/407; C07D 487/04

(52) U.S. Cl. 514/413; 548/452

(57) ABSTRACT

Tetrahydro-pyrrolizinone compounds which mediate the activity of LFA-1 with its ligands involved in cell adhesion, migration and activation.

The present invention relates to tetrahydro-pyrrolizinone compounds which are pharmaceutically active.

Inflammatory recruitment of leukocytes is governed by dynamic interactions between integrins and endothelial immunoglobulin superfamily (IgSF) proteins, such as ICAM-1 (intercellular adhesion molecule-1), ICAM-2, ICAM-3 and the IgSF member junctional adhesion molecule 1 (JAM-1), all of which have been identified to be a ligand of the beta(2) integrin lymphocyte function-associated antigen 1 (LFA-1). Compounds which mediate, e.g. inhibit, interaction of LFA-1 and its ligands involved in cell adhesion, migration and activation have been found to constitute a therapeutic approach to inflammation and autoimmune diseases.

Now surprisingly compounds have been found which mediate, e.g. inhibit the activity of LFA-1 with its ligands involved in cell adhesion, migration and activation. In one aspect the present invention provides a compound of formula

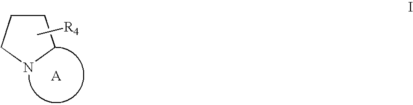

wherein

ring A is a group of formula

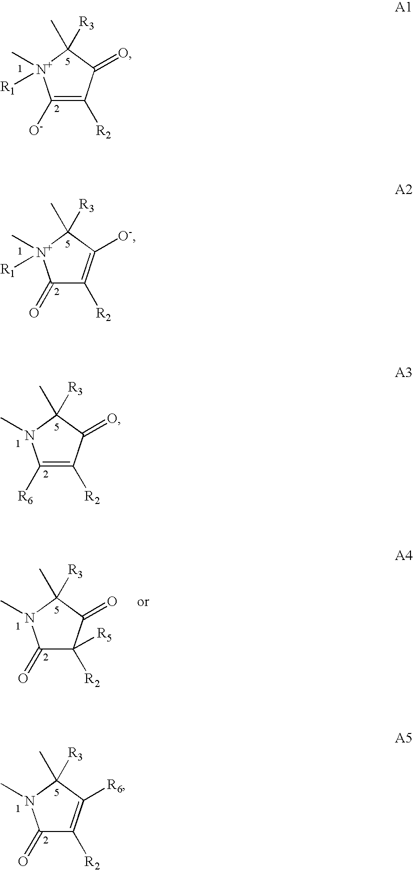

R₁ is

alkyl, e.g. (C₁₋₁₈)alkyl, such as (C₁₋₆)alkyl; e.g. (C₁₋₄)alkyl; alkenyl, e.g. (C₂₋₁₈)alkenyl, such as (C₂₋₆)alkenyl, alkynyl, (C₂₋₁₈)alkynyl, such as (C₂₋₄)alkynyl, or alkyl, alkenyl or alkynyl substituted by
alkyoxy, e.g. (C₁₋₄)alkoxy,
a silyl or silyloxy group, such as trialkylsilyl, e.g. tri(C₁₋₆)alkylsilyl, such as trimethylsilyl or tri(C₁₋₆)alkylsilyloxy, such as (tert.butyl)(dimethyl)silyloxy,
cycloalkyl, e.g. (C₃₋₁₈)cycloalkyl,
aryl, e.g. (C₆₋₁₈)aryl, such as (C₆₋₁₂)aryl, or
heterocyclyl,

R₂ is

cycloalkyl, e.g. (C₃₋₁₈)cycloalkyl,

aryl, e.g. (C₃₋₁₈)aryl, such as (C₆₋₁₂)aryl, or heterocyclyl,

R₃ is hydrogen or optionally substituted
alkyl, e.g. (C₁₋₈)alkyl, alkenyl, such as (C₂₋₈)alkenyl, alkynyl, such as (C₂₋₈)alkynyl,, e.g. unsubstituted alkyl, alkenyl or alkynyl, or substituted alkyl, alkenyl or alkynyl, such as
alkyl, alkenyl or alkynyl substituted by
cycloalkyl, e.g. (C₃₋₁₈)cycloalkyl,
aryl, e.g. (C₃₋₁₈)aryl, such as (C₆₋₁₂)aryl, or
heterocyclyl,

R₄ is

trialkylsilyloxy, e.g. tri(C₁₋₆)alkylsilyloxy, N₃, amino, alkylamino, such as (C₁₋₈)alkylamino, dialkylamino, such as (C₁₋₈)dialkylamino, cycloalkylamino, such as (C₃₋₈)cycloalkylamino, acylamino, e.g. including (C₂₋₁₈)acylamino, such as (C₁₋₈)alkylcarbonylamino, (C₆₋₁₂)arylcarbonylamino, (C₃₋₈)cycloalkylcarbonylamino, and heterocyclylcarbonylamino; or (acyl)(alkyl)-amino, such as ((C₂₋₁₈)acyl)-((C₁₋₄)alkyl))-amino, e.g. alkyl N-methyl-N-methylcarbonyl-amino, N-benzyl-N-methylcarbonyl-amino, N-ethyl-N-methylcarbonyl-amino, sulfonylamino, such as (C₁₋₄)alkysulfonylamino, (C₆₋₁₂)arylsulfonylamino, (C₃₋₈)cyclohexylsulfonylamino, or R₄ is heterocyclyl comprising at least one nitrogen atom as a heteroatom and being bound via that nitrogen atom to a compound of formula 1, R₅ is hydrogen, unsubstituted alkyl, e.g. (C₁₋₁₈)alkyl, such as (C₁₋₆)alkyl; e.g. (C₁₋₄)alkyl; alkenyl, e.g. (C₂₋₁₈)alkenyl, such as (C₂₋₅)alkenyl, alkynyl, (C₂₋₁₈)alkynyl, such as (C₂₋₄)alkynyl, or alkyl, alkenyl or alkynyl substituted by
alkyoxy, e.g. (C₁₋₄)alkoxy,
a silyl or silyloxy group, such as trialkylsilyl, e.g. tri(C₁₋₆)alkylsilyl, such as trimethylsilyl or tri(C₁₋₆)alkylsilyloxy, such as (tert.butyl)(dimethyl)silyloxy,
cycloalkyl, e.g. (C₃₋₁₈)cycloalkyl,
aryl, e.g. (C₃₋₁₈)aryl, such as (C₆₋₁₂)aryl, or
heterocyclyl,

R₆ is

OR₇or SR₇,

R₇ is hydrogen, a group (SO)₂—R₉, wherein R₉ is (C₁₋₄)alkyl or (C₆₋₁₂)aryl, alkyl, e.g. (C₁₋₁₈)alkyl, such as (C₁₋₆)alkyl; e.g. (C₁₋₄)alkyl; alkenyl, e.g. (C₂₋₁₈)alkenyl, such as (C₂₋₆)alkenyl, alkynyl, (C₂₋₁₈)alkynyl, such as (C₂₋₄)alkynyl, e.g. wherein alkyl, alkenyl or alkynyl is unsubstituted, or substituted
alkyoxy, e.g. (C₁₋₄)alkoxy,
a silyl or silyloxy group, such as trialkylsilyl, e.g. tri(C₁₋₆)alkylsilyl, such as trimethylsilyl or tri(C₁₋₆)alkylsilyloxy, e.g. (tert.butyl)(dimethyl)silyloxy,
cycloalkyl, e.g. (C₃₋₁₈)cycloalkyl,
aryl, e.g. (C₆₋₁₈)aryl, such as (C₆₋₁₂)aryl, or
heterocyclyl,

or R₇ is COR₈ or CSR₈,

R₈ is

alkyl, e.g. (C₁₋₁₈)alkyl, such as (C₁₋₆)alkyl; e.g. (C₁₋₄)alkyl; alkenyl, e.g. (C₂₋₁₈)alkenyl, such as (C₂₋₆)alkenyl, alkynyl, (C₂₋₁₈)alkynyl, such as (C₂₋₄)alkynyl, wherein alkyl, alkenyl or alkynyl is unsubstituted or substituted by
cycloalkyl, e.g. (C₃₋₁₈)cycloalkyl,
aryl, e.g. (C₆₋₁₈)aryl, such as (C₆₋₁₂)aryl, or
heterocyclyl,

cycloalkyl, e.g. (C₃₋₁₈)cycloalkyl, aryl, e.g. (C₆₋₁₈)aryl, such as (C₆₋₁₂)aryl, or heterocyclyl, e.g. wherein

cycloalkyl, aryl or heterocyclyl are unsubstituted or substituted, e.g. unsubstituted or substituted by one or more groups such as conventional in organic chemistry, e.g. including alkyl, e.g. (C₁₋₆)alkyl, alkenyl, such as (C₂₋₁₆)alkenyl, alkynyl, such as (C₂₋₁₆)alkynyl, cycloalkyl, such as (C₃₋₈)cycloalkyl, aryl, such as phenyl, aralkyl, such as benzyl, heterocyclyl, haloalkyl, such as (C₁₋₄)haloalkyl, alkoxy, such as (C₁₋₈)alkoxy, aryloxy, such as phenoxy, oxo,

acyl, such as (C₂₋₁₃)acyl, e.g. including alkylcarbonyl, e.g. (C₁₋₄)alkylcarbonyl, arylcarbonyl, e.g. phenylcarbonyl, heterocyclylcarbonyl, (C₂₋₁₃)acyloxy, e.g. including alkylcarbonyloxy, e.g. (C₁₋₄)alkylcarbonyloxy, arylcarbonyloxy, e.g. phenylcarbonyloxy, heterocyclylcarbonyloxy,

amino, such as unsubstituted amino and substituted amino, e.g. (C₁₋₆)alkylamino, (C₁₋₆)dialkylamino,

acylamino, such as (C₂₋₁₃)acylamino, including (C₁₋₄)alkylaminocarbonyl,

phenylaminocarbonyl, benzylaminocarbonyl, heterocyclylaminocarbonyl,

nitro, cyano, halogen, alkylsulfonyl, e.g. (C₁₋₄)alkylsulfonyl, arylsulfonyl, such as tolylsulfonyl, tri(C₁₋₆) alkylsilyl or tri(C₁₋₆)silyloxy,

e.g. wherein heterocyclyl comprises
aliphatic and aromatic heterocyclyl, preferably aromatic heterocyclyl,
3 to 8 ring members, such as 5 to 6 ring members,
1 to 4 heteroatoms selected from N, O, S,
fused heterocyclyl, such as heterocyclyl fused with another ring (system), with the proviso that, if R₄ is heterocyclyl, said heterocyclyl comprising at least one nitrogen atom as a heteroatom and being bound via that nitrogen atom to a compound of formula I.

In a compound of formula I R₄ is attached to a CH₂ group in the ring of formula I which is not ring A, namely to a CH₂ group in the pyrrolidinyl ring of formula I.

Preferably substitutents of cycloalkyl, aryl or heterocyclyl include (C₁₋₁₆)alkyl, (C₃₋₈)cyclohexyl, (C₁₋₄)alkylcarbonyl, such as methylcarbonyl, (C₁₋₄)alkoxycarbonyl, e.g. methoxycarbonyl, halogen, cyano, aminocarbonyl, heterocyclyl comprising 5 to 6 ring members and 1 to 4 heteroatoms selected from N, O, S, e.g. including aromatic heterocyclyl and aliphatic heterocyclyl, preferably aromatic heterocycyl, tri(C₁₋₆)alkylsilyl, or tri(C₁₋₆)alkylsilyloxy.

Preferably R₁ is

(C₁₋₆)alkyl, such as (C₁₋₄)alkyl, e.g. methyl or ethyl, (C₂₋₆)alkenyl, such as propenyl, e.g. propen-3yl, (C₂₋₆)alkynyl, such as (C₂₋₄)alkynyl, e.g. propynyl, such as propyn-3-yl, wherein (C₁₋₆)alkyl, (C₂₋₆)alkenyl, or (C₂₋₄)alkynyl is unsubstituted or substituted by
(C₆₋₁₈)aryl, such as (C₆₋₁₂) aryl, e.g. phenyl,
a silyl or silyloxy group, such as trialkylsilyl, e.g. tri(C₁₋₆)alkylsilyl, such as trimethylsilyl or tri(C₁₋₆)alkylsilyloxy, e.g. (tert.butyl)(dimethyl)silyloxy,

e.g. wherein aryl is unsubstituted or substituted, e.g. unsubstituted or substituted (C₁₋₁₆)alkyl, (C₃₋₈)cyclohexyl, (C₁₋₄)alkylcarbonyl, such as methylcarbonyl, (C₁₋₄)alkoxycarbonyl, e.g. methoxycarbonyl, halogen, cyano, aminocarbonyl, heterocyclyl comprising 5 to 6 ring members and 1 to 4 heteroatoms selected from N, O, S, e.g. including aromatic heterocyclyl and aliphatic heterocyclyl, preferably aromatic heterocyclyl, tri(C₁₋₆)alkylsilyl or tri(C₁₋₆)alkylsilyloxy;

e.g. R₁ is methyl, ethyl, propen-3yl, 1-(trimethylsilyl)propyn-3yl, or cyaonphenylmethyl, such as 4-cyanophenylmethyl.

Preferably R₂ is optionally substituted aryl, e.g. (C₆₋₁₈)aryl, such as phenyl, e.g. unsubstituted aryl, or aryl substituted by (C₁₋₁₆)alkyl, (C₃₋₈)cyclohexyl, (C₁₋₄)alkylcarbonyl, such as methylcarbonyl, (C₁₋₄)alkoxycarbonyl, e.g. methoxycarbonyl, halogen, cyano, aminocarbonyl, heterocyclyl comprising 5 to 6 ring members and 1 to 4 heteroatoms selected from N, O, S, e.g. including aromatic heterocyclyl and aliphatic heterocyclyl; preferably aromatic heterocyclyl, tri(C₁₋₆)alkylsilyl or tri(C₁₋₆)alkylsilyloxy; such as halogen, e.g. R₂ is dihalophenyl, such as 3,5-dichlorophenyl.

Preferably R₃ is hydrogen, (C₂₋₆)alkenyl, (C₂₋₆)alkynyl, or (C₆₋₁₂)aryl(C₁₋₄)alkyl, e.g. wherein aryl is unsubstituted aryl or substituted by (C₁₋₁₆)alkyl, (C₃₋₈)cyclohexyl, (C₁₋₄)alkylcarbonyl, such as methylcarbonyl, (C₁₋₄)alkoxycarbonyl, e.g. methoxycarbonyl, halogen, cyano, aminocarbonyl, heterocyclyl comprising 5 to 6 ring members and 1 to 4 heteroatoms selected from N, O, S, e.g. including aromatic heterocyclyl and aliphatic heterocyclyl; preferably aromatic heterocyclyl, tri(C₁₋₆)alkylsilyl or tri(C₁₋₆)alkylsilyloxy;

such as phenyl substituted by halogen, cyano or aromatic heterocyclyl,

e.g. R₃ is hydrogen, propadienyl, e.g. propa-1,2-dienyl, propynyl, e.g. propyn-3-yl, benzyl, cyanophenylmethyl, e.g. 4-cyanophenylmethyl, halophenylmethyl, e.g. bromophenylmethyl, such as 4-bromophenylmethyl or pyrimidinophenylmethyl.

Preferably R₄ is

tri(C₁₋₆)alkylsilyloxy, N₃, amino, (C₁₋₄)alkylcarbonylamino, sulfonylamino, such as (C₁₋₄)alkylsulfonylamino, (C₆₋₁₂)arylcarbonylamino, N-((C₁₋₄)alkylcarbonyl)-N-((C₁₋₄)alkyl)-amino, N-((C₆₋₁₂)aryl(C₁₋₄)alkylcarbonyl)-N-((C₁₋₄)alkyl)-amino, or aromatic heterocyclyl comprising 5 or 6 ring members and at least one nitrogen atom and being bound via that nitrogen atom to a compound of formula I,

e.g. R₄ is (tert-butyl)(dimethyl)silyloxy, N₃, amino, methylcarbonylamino, tert-butylcarbonylamino, phenylcarbonylamino, N-methylcarbonyl-N-methyl-amino, N-benzyl-N-methylcarbonyl-amino, N-ethyl-N-methylcarbonyl-amino, methylsulfonylamino, or unsubstituted or substituted triazolyl bound to a compound of formula I via a nitrogen heteroatom, e.g. triazolyl substituted by isobutyl, isopentyl, n-tridecanyl, cyclopentyl, phenyl, methyloxycarbonyl, 1-methyl-propylcarbonyl, isorpoylcarbonyl, dodecanylcarbonyl, oxo, halogen, e.g. iodo, tri(C₁₋₄)alkylylsilyl, such as trimethylsilyl, or aromatic heterocyclyl comprising 5 or 6 ring members and 1 to 4 heteroatoms, e.g. 1, selected from N, O, S, e.g. N, such as pyridinyl,

Preferably R₅ is hydrogen or (C₁₋₄)alkyl substituted by phenyl, wherein phenyl is unsubstituted or substituted, e.g. wherein aryl is unsubstituted aryl, or aryl substituted by (C₁₋₁₆)alkyl, (C₃₋₈)cyclohexyl, (C₁₋₄)alkylcarbonyl, such as methylcarbonyl, (C₁₋₄)alkoxycarbonyl, e.g. methoxycarbonyl, halogen, cyano, aminocarbonyl, heterocyclyl comprising 5 to 6 ring members and 1 to 4 heteroatoms selected from N, O, S, e.g. including aromatic heterocyclyl and aliphatic heterocyclyl; tri(C₁₋₆)alkylsilyl or tri(C₁₋₆)alkylsilyloxy; such as phenyl substituted by halogen, cyano or aromatic heterocyclyl,

e.g. R₅ is cyanophenyl, such as 4-cyanophenyl.

Preferably R₇ is OR₇.

Preferably R₇ is

hydrogen, (C₁₋₈)alkyl, (C₁₋₈)alkoxy(C₂₋₈)alkyl, (C₂₋₈)alkenyl, (C₂₋₈)alkynyl, (C₂₋₈)alkynyl substituted by tri(C₁₋₄)alkylsilyl, (C₁₋₄)alkyl substituted by phenyl, a group (SO)₂—R₉, or COR₈, e.g. wherein aryl is unsubstituted aryl or substituted by (C₁₋₁₆)alkyl, (C₃₋₈)cyclohexyl, (C₁₋₄)alkylcarbonyl, such as methylcarbonyl, (C₁₋₄)alkoxycarbonyl, e.g. methoxycarbonyl, halogen, cyano, aminocarbonyl, heterocyclyl comprising 5 to 6 ring members and 1 to 4 heteroatoms selected from N, O, S, e.g. including aromatic heterocyclyl and aliphatic heterocyclyl; tri(C₁₋₆)alkylsilyl or tri(C₁₋₆)alkylsilyloxy; such as phenyl substituted by halogen, cyano or aromatic heterocyclyl,

e.g. R₇ is hydrogen, methyl, propenyl, e.g. propen-3-yl, trimethylsilyl-propynyl, e.g. 1-trimethylsilyl-propyn-3-yl, cyanophenylmethyl, such as 4-cyanophenylmethyl, tolylsulfonyl or methylcarbonyl.

Preferably R₈ is (C₁₋₄)alkyl; e.g. methyl.

Preferably R₉ is (C₆₋₁₂)aryl, e.g. phenyl, such as unsubstituted or substituted aryl, e.g. aryl substituted by (C₁₋₄)alkyl, such as tolyl.

The present invention includes a compound of formula 1, wherein one, more or all of the residues defined have a preferred meaning as defined above, and the other residues have the meaning as defined above.

In a compound of formula I each single substituent defined may be a preferred substituent, e.g. independently from the other subsitutents defined.

In another aspect the present invention provides a compound of formula 1, wherein ring A is as defined above,

R₁ is methyl, ethyl, propen-3yl, 1-(trimethylsilyl)propyn-3yl, or cyaonphenylmethyl,

R₂ is dihalophenyl,

R₃ is hydrogen, propadienyl, propynyl, benzyl, cyanophenylmethyl, halophenylmethyl or pyrimidinophenylmethyl,

R₄ is (tert-butyl)(dimethyl)silyloxy, N₃, amino, methylcarbonylamino, tert-butylcarbonylamino, phenylcarbonylamino, N-methylcarbonyl-N-methyl-amino, N-benzyl-N-methylcarbonyl-amino, N-ethyl-N-methylcarbonyl-amino, methylsulfonylamino, or substituted triazolyl bound to a compound of formula I via a nitrogen heteroatom, wherein triazolyl is substituted by isobutyl, isopentyl, n-tridecanyl, cyclopentyl, phenyl, methyloxycarbonyl, 1-methyl-propylcarbonyl, isorpoylcarbonyl, dodecanylcarbonyl, oxo, halogen, tri(C₁₋₄)alkylylsilyl, or pyridinyl,

R₅ is cyanophenyl,

R₆ is OR₇, and

R₇ is hydrogen, methyl, propenyl, trimethylsilyl-propynyl, cyanophenylmethyl, tolylsulfonyl or methylcarbonyl.

In a compound of formula I, the substituent R₄ is preferably in the position as set out in formula

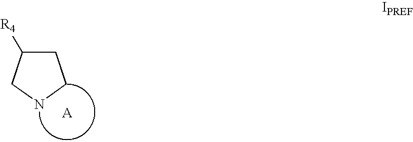

Compounds of formula I wherein ring A is a group of formula A1 or formula A2, or compounds of formula I wherein ring A is a group of formula A3 or formula A5 wherein R₆ is hydroxy may be present in a chemical equilibrium.

In another aspect the present invention provides a compound selected from the group consisting of

N-[7a-(4-Cyano-benzyl)-6-(3,5-dichloro-phenyl)-7-hydroxy-5-oxo-2,3,5,7a-tetrahydro-1H-pyrrolizin-2-yl]-acetamide, such as N-[(2S,7aS)-7a-(4-cyano-benzyl)-6-(3,5-dichloro-phenyl)-7-hydroxy-5-oxo-2,3,5,7a-tetrahydro-1H-pyrrolizin-2-yl]-acetamide;

N-[7a-(4-Cyano-benzyl)-6-(3,5-dichloro-phenyl)-7-methoxy-5-oxo-2,3,5,7a-tetrahydro-1H-pyrrolizin-2-yl]-acetamide, such as N-[(2S,7aS)-7a-(4-cyano-benzyl)-6-(3,5-dichloro-phenyl)-7-methoxy-5-oxo-2,3,5,7a-tetrahydro-1H-pyrrolizin-2-yl]-acetamide;

N-[7a-(4-Cyano-benzyl)-6-(3,5-dichloro-phenyl)-5-methoxy-7-oxo-2,3,7,7a-tetrahydro-1H-pyrrolizin-2-yl]-acetamide, such as N-[(2S,7aS)-7a-(4-cyano-benzyl)-6-(3,5-dichloro-phenyl)-5-methoxy-7-oxo-2,3,7,7a-tetrahydro-1H-pyrrolizin-2-yl]-acetamide;

7a-(4-Bromo-benzyl)-6-(tert-butyl-dimethyl-silanyloxy)-2-(3,5-dichloro-phenyl)-3-methoxy-5,6,7,7a -tetrahydro-pyrrolizin-1-one, such as (6R,7aS)-7a-(4-bromo-benzyl)-6-(tert-butyl-dimethyl-silanyloxy)-2-(3,5-dichloro-phenyl)-3-methoxy-5,6,7,7a-tetrahydro-pyrrolizin-1-one;

7a-(4-Bromo-benzyl)-6-(tert-butyl-dimethyl-silanyloxy)-2-(3,5-dichloro-phenyl)-3-methoxy-5,6,7,7a-tetrahydro-pyrrolizin-1-one, such as(6R,7aS)-7a-(4-bromo-benzyl)-6-(tert-butyl-dimethyl-silanyloxy)-2-(3,5-dichloro-phenyl)-3-methoxy-5,6,7,7a-tetrahydro-pyrrolizin-1-one;

N-[7a-(4-Bromo-benzyl)-6-(3,5-dichloro-phenyl)-5-methoxy-7-oxo-2,3,7,7a-tetrahydro-1H-pyrrolizin-2-yl]-acetamide, such as N-[(2S,7aS)-7a-(4-bromo-benzyl)-6-(3,5-dichloro-phenyl)-5-methoxy-7-oxo-2,3,7,7a-tetrahydro-1H-pyrrolizin-2-yl]-acetamide;

6-Azido-2-(3,5-dichloro-phenyl)-3-methoxy-7a-(4-pyrimidin-5-yl-benzyl)-5,6,7,7a-tetrahydro-pyrrolizin-1-one, such as (6S,7aS)-6-azido-2-(3,5-dichloro-phenyl)-3-methoxy-7a-(4-pyrimidin-5-yl-benzyl)-5,6,7,7a-tetrahydro-pyrrolizin-1-one;

6-(tert-Butyl-dimethyl-silanyloxy)-3-methoxy-2-phenyl-5,6,7,7a-tetrahydro-pyrrolizin-1-one, such as (6R,7aS)-6-(tert-Butyl-dimethyl-silanyloxy)-3-methoxy-2-phenyl-5,6,7,7a-tetrahydro-pyrrolizin-1-one and (6R,7aR)-6-(tert-butyl-dimethyl-silanyloxy)-3-methoxy-2-phenyl-5,6,7,7a-tetrahydro-pyrrolizin-1-one;

7a-(4-Bromo-benzyl)-6-(tert-butyl-dimethyl-silanyloxy)-1-hydroxy-2-phenyl-5,6,7,7a-tetrahydro-pyrrolizin-3-one, such as (6R,7aR)-7a-(4-bromo-benzyl)-6-(tert-butyl-dimethyl-silanyloxy)-1-hydroxy-2-phenyl-5,6,7,7a-tetrahydro-pyrrolizin-3-one;

6-(tert-Butyl-dimethyl-silanyloxy)-1-methoxy-2-phenyl-5,6,7,7a-tetrahydro-pyrrolizin-3-one, such as (6R,7aR)-6-(tert-butyl-dimethyl-silanyloxy)-1-methoxy-2-phenyl-5,6,7,7a-tetrahydro-pyrrolizin-3-one;

6-(tert-Butyl-dimethyl-silanyloxy)-1-hydroxy-2-phenyl-5,6,7,7a-tetrahydro-pyrrolizin-3-one, such as (6R,7aR)-6-(tert-Butyl-dimethyl-silanyloxy)-1-hydroxy-2-phenyl-5,6,7,7a-tetrahydro-pyrrolizin-3-one; or (6R,7aS)-6-(tert-Butyl-dimethyl-silanyloxy)-1-hydroxy-2-phenyl-5,6,7,7a-tetrahydro-pyrrolizin-3-one;

4-[6-(tert-Butyl-dimethyl-silanyloxy)-3-oxo-2-phenyl-5,6,7,7a-tetrahydro-3H-pyrrolizin-1-yloxymethyl]-benzonitrile, such as 4-[(6R,7aR)-6-(tert-Butyl-dimethyl-silanyloxy)-3-oxo-2-phenyl-5,6,7,7a-tetrahydro-3H-pyrrolizin-1-yloxymethyl]benzonitrile;

A compound of formula

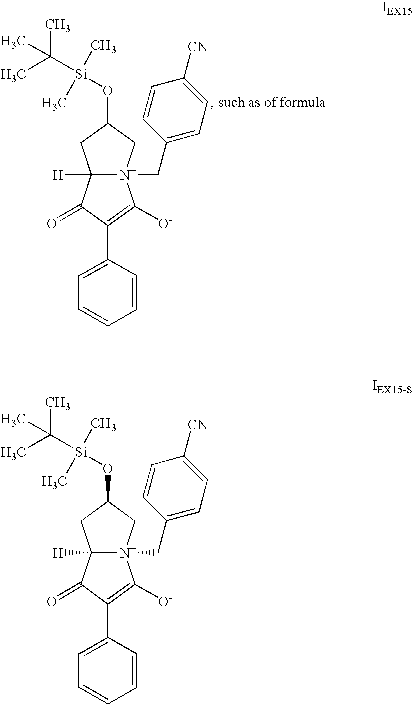

4-[6-(tert-Butyl-dimethyl-silanyloxy)-1,3-dioxo-2-phenyl-hexahydro-pyrrolizin-2-ylmethyl]-benzonitrile, such as 4-[(6S,7aS)-6-(tert-butyl-dimethyl-silanyloxy)-1,3-dioxo-2-phenyl-hexahydro-pyrrolizin-2-ylmethyl]-benzonitrile;

7a-(4-Bromo-benzyl)-6-(tert-butyl-dimethyl-silanyloxy)-2-(3,5-dichloro-phenyl)-1-hydroxy-5,6,7,7a-tetrahydro-pyrrolizin-3-one, such as (6R,7aS)-7a-(4-bromo-benzyl)-6-(tert-butyl-dimethyl-silanyloxy)-2-(3,5-dichloro-phenyl)-1-hydroxy-5,6,7,7a-tetrahydro-pyrrolizin-3-one;

7a-(4-Bromo-benzyl)-6-(tert-butyl-dimethyl-silanyloxy)-2-(3,5-dichloro-phenyl)-1-methoxy-5,6,7,7a-tetrahydro-pyrrolizin-3-one, such as (6R,7aS)-7a-(4-bromo-benzyl)-6-(ter t-butyl-dimethyl-silanyloxy)-2-(3,5-dichloro-phenyl)-1-methoxy-5,6,7,7a-tetrahydro-pyrrolizin-3-one;

7a-(4-Bromo-benzyl)-6-(tert-butyl-dimethyl-silanyloxy)-2-(3,5-dichloro-phenyl)-1-(4-methoxy-butoxy)-5,6,7,7a-tetrahydro-pyrrolizin-3-one, such as (6R,7aS)-7a-(4-Bromo-benzyl)-6-(ter t-butyl-dimethyl-silanyloxy)-2-(3,5-dichloro-phenyl)-1-(4-methoxy-butoxy)-5,6,7,7a-tetrahydro-pyrrolizin-3-one;

6-(tert-Butyl-dimethyl-silanyloxy)-1-methoxy-2-phenyl-5,6,7,7a-tetrahydro-pyrrolizin-3-one, such as (6R,7aS)-6-(tert-Butyl-dimethyl-silanyloxy)-1-methoxy-2-phenyl-5,6,7,7a-tetrahydro-pyrrolizin-3-one;

Toluene4-sulfonic acid 6-(tert-butyl-dimethyl-silanyloxy)-3-oxo-2-phenyl-5,6,7,7a-tetrahydro-3H-pyrrolizin-1-yl ester, such as toluene-4-sulfonic acid (6R,7aS)-6-(tert-butyl-dimethyl-silanyloxy)-3-oxo-2-phenyl-5,6,7,7a-tetrahydro-3H-pyrrolizin-1-yl ester;

4-[6-(tert-Butyl-dimethyl-silanyloxy)-3-oxo-2-phenyl-5,6,7,7a-tetrahydro-3H-pyrrolizin-1-yloxymethyl]-benzonitrile, such as 4-[(6R,7aS)-6-(tert-butyl-dimethyl-silanyloxy)-3-oxo-2-phenyl-5,6,7,7a-tetrahydro-3H-pyrrolizin-1-yloxymethyl]-benzonitrile;

1-Allyloxy-6-(tert-butyl-dimethyl-silanyloxy)-2-phenyl-5,6,7,7a-tetrahydro-pyrrolizin-3-one, such as(6R,7aS)-1-Allyloxy-6-(tert-butyl-dimethyl-silanyloxy)-2-phenyl-5,6,7,7a-tetrahydro-pyrrolizin-3-one;

6-(tert-Butyl-dimethyl-silanyloxy)-2-phenyl-1-(3-trimethylsilanyl-prop-2-ynyloxy)-5,6,7,7a-tetra hydro-pyrrolizin-3-one, such as (6R,7aS)-6-(tert-butyl-dimethyl-silanyloxy)-2-phenyl-1-(3-trimethylsilanyl-prop-2-ynyloxy)-5,6,7,7a-tetrahydro-pyrrolizin-3-one;

6-Azido-7a-(4-bromo-benzyl)-2-(3,5-dichloro-phenyl)-1-hydroxy-5,6,7,7a-tetrahydro-pyrrolizin-3-one, such as (6S,7aS)-6-azido-7a-(4-bromo-benzyl)-2-(3,5-dichloro-phenyl)-1-hydroxy-5,6,7,7a-tetrahydro-pyrrolizin-3-one;

6-Amino-7a-benzyl-2-(3,5-dichloro-phenyl)-1-hydroxy-5,6,7,7a-tetrahydro-pyrrolizin-3-one, such as (6S,7aS)-6-amino-7a-benzyl-2-(3,5-dichloro-phenyl)-1-hydroxy-5,6,7,7a-tetrahydro-pyrrolizin-3-one;

Acetic acid 6-acetylamino-7a-benzyl-2-(3,5-dichloro-phenyl)-3-oxo-5,6,7,7a-tetrahydro-3H-pyrrolizin-1-yl ester, such as acetic acid (6S,7aS)-6-acetylamino-7a-benzyl-2-(3,5-dichloro-phenyl)-3-oxo-5,6,7,7a-tetrahydro-3H-pyrrolizin-1-yl ester;

N-[7a-(4-Bromo-benzyl)-6-(3,5-dichloro-phenyl)-7-hydroxy-5-oxo-2,3,5,7a-tetrahydro-1H-pyrrolizin-2-yl]-acetamide, such as N-[(2S,7aS)-7a-(4-bromo-benzyl)-6-(3,5-dichloro-phenyl)-7-hydroxy-5-oxo-2,3,5,7a-tetrahydro-1H-pyrrolizin-2-yl]-acetamide;

N-[7a-(4-Bromo-benzyl)-6-(3,5-dichloro-phenyl)-7-methoxy-5-oxo-2,3,5,7a-tetrahydro-1H-pyrrolizin-2-yl]-acetamide, such as N-[(2S,7aS)-7a-(4-bromo-benzyl)-6-(3,5-dichloro-phenyl)-7-methoxy-5-oxo-2,3,5,7a-tetrahydro-1H-pyrrolizin-2-yl]-acetamide;

6-Azido-2-(3,5-dichloro-phenyl)-1-methoxy-7a-(4-pyrimidin-5-yl-benzyl)-5,6,7,7a-tetrahydro-pyrrolizin-3-one, such as (6S,7aS)-6-Azido-2-(3,5-dichloro-phenyl)-1-methoxy-7a-(4-pyrimidin-5-yl-benzyl)-5,6,7,7a-tetrahydro-pyrrolizin-3-one;

6-(tert-Butyl-dimethyl-silanyloxy)-2-(3,5-dichloro-phenyl)-7a-propa-1,2-dienyl-5,6,7,7a-tetrahydro-pyrrolizin-3-one, such as (6R,7aR)-6-(tert-butyl-dimethyl-silanyloxy)-2-(3,5-dichloro-phenyl)-7a-propa-1,2-dienyl-5,6,7,7a-tetrahydro-pyrrolizin-3-one

6-(tert-Butyl-dimethyl-silanyloxy)-2-(3,5-dichloro-phenyl)-1-hydroxy-7a-prop-2-ynyl-5,6,7,7a-tetrahydro-pyrrolizin-3-one, such as (6S,7aS)-6-(tert-butyl-dimethyl-silanyloxy)-2-(3,5-dichloro-phenyl)-1-hydroxy-7a-prop-2-ynyl-5,6,7,7a-tetrahydro-pyrrolizin-3-one;

A compound of formula

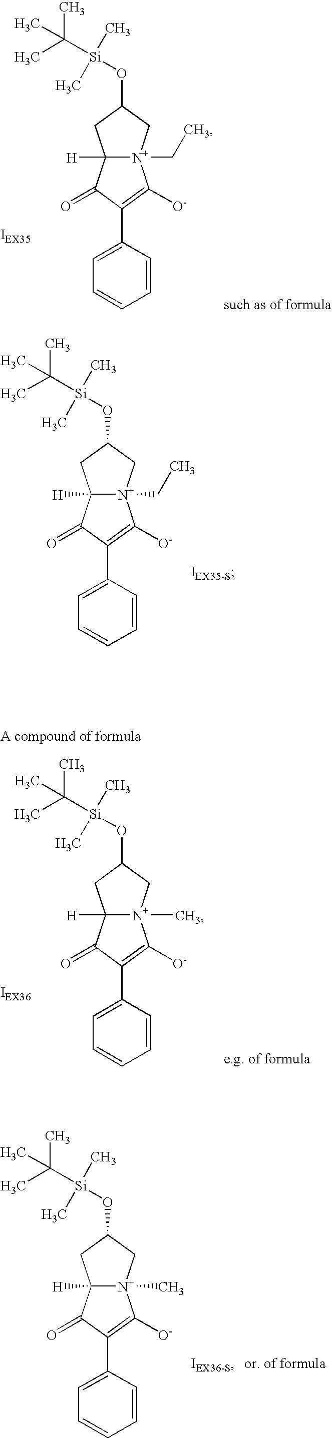

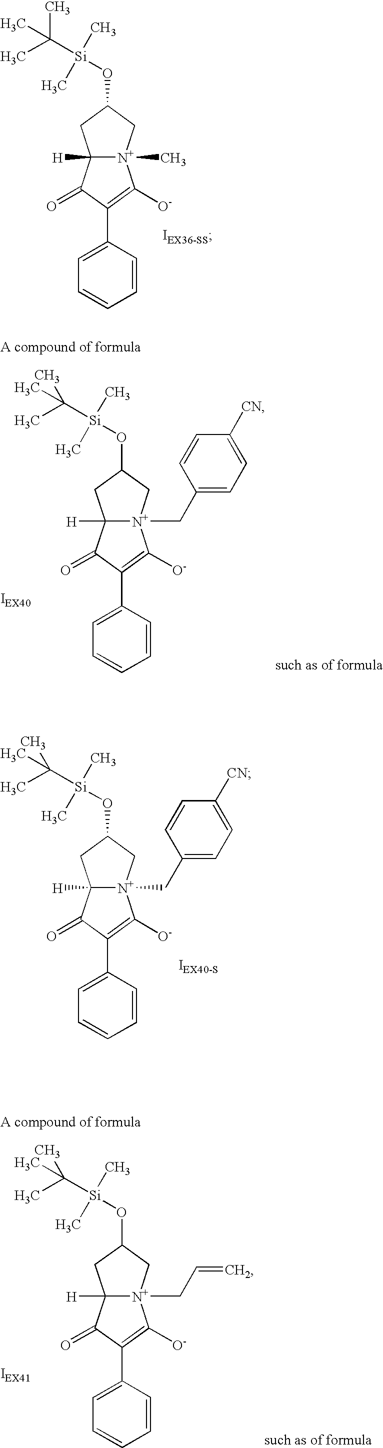

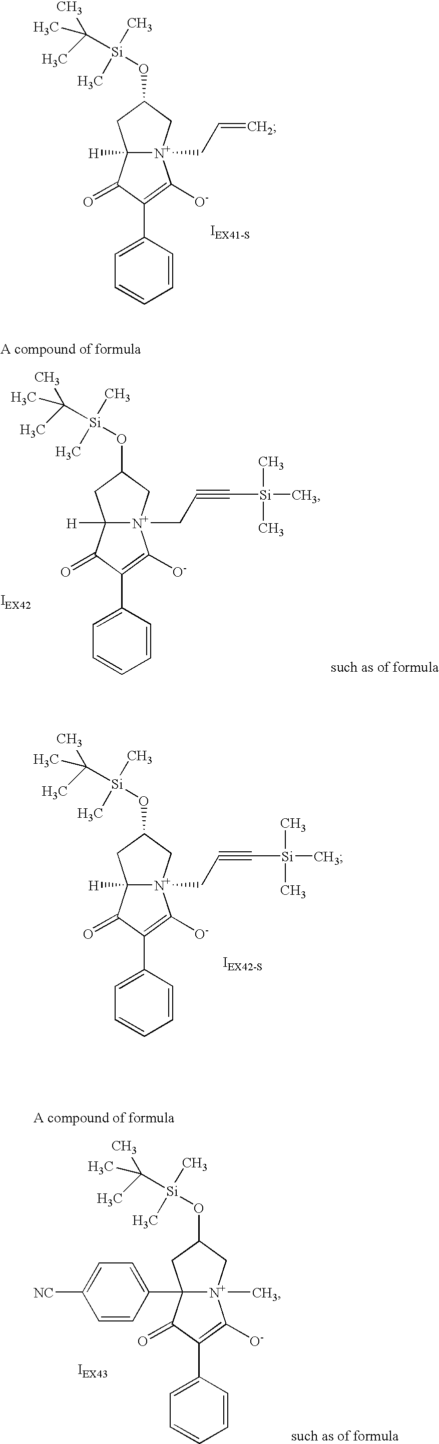

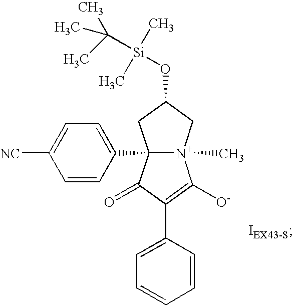

4-[2-Azido-6-(3,5-dichloro-phenyl)-7-hydroxy-5-oxo-2,3-dihydro-1H,5H-pyrrolizin-7a-ylmethyl]-benzonitrile, such as 4-[(2S,7aS)-2-Azido-6-(3,5-dichloro-phenyl)-7-hydroxy-5-oxo-2,3-dihydro-1H,5H-pyrrolizin-7a-ylmethyl]-benzonitrile;

4-[2-Amino-6-(3,5-dichloro-phenyl)-7-methoxy-5-oxo-2,3-dihydro-1H,5H-pyrrolizin-7a-ylmethyl]-benzonitrile, such as 4-[(2S,7aS)-2-amino-6-(3,5-dichloro-phenyl)-7-methoxy-5-oxo-2,3-dihydro-1H,5H-pyrrolizin-7a-ylmethyl]-benzonitrile;

N-[7a-(4-Cyano-benzyl)-6-(3,5-dichloro-phenyl)-7-methoxy-5-oxo-2,3,5,7a-tetrahydro-1H-pyrrolizin-2-yl]-methanesulfonamide, such as N-[(2S,7aS)-7a-(4-cyano-benzyl)-6-(3,5-dichloro-phenyl)-7-methoxy-5-oxo-2,3,5,7a-tetrahydro-1H-pyrrolizin-2-yl]-methanesulfonamide;

N-[7a-(4-Cyano-benzyl)-6-(3,5-dichloro-phenyl)-7-methoxy-5-oxo-2,3,5,7a-tetrahydro-1H-pyrrolizin-2-yl]-2,2-dimethyl-propionamide, such as N-[(2S,7aS)-7a-(4-cyano-benzyl)-6-(3,5-dichloro-phenyl)-7-methoxy-5-oxo-2,3,5,7a-tetrahydro-1H-pyrrolizin;

N-[7a-(4-Cyano-benzyl)-6-(3,5-dichloro-phenyl)-7-methoxy-5-oxo-2,3,5,7a-tetrahydro-1H-pyrrolizin-2-yl]-benzamide, such as N-[(2S,7aS)-7a-(4-cyano-benzyl)-6-(3,5-dichloro-phenyl)-7-methoxy-5-oxo-2,3,5,7a-tetrahydro-1H-pyrrolizin-2-yl]-benzamide;

N-[7a-(4-Cyano-benzyl)-6-(3,5-dichloro-phenyl)-7-methoxy-5-oxo-2,3,5,7a-tetrahydro-1H-pyrrolizin-2-yl]-N-methyl-acetamide, such as N-[(2S,7aS)-7a-(4-cyano-benzyl)-6-(3,5-dichloro-phenyl)-7-methoxy-5-oxo-2,3,5,7a-tetrahydro-1H-pyrrolizin-2-yl]-N-methyl-acetamide;

4-[2-Acetylamino-6-(3,5-dichloro-phenyl)-7-methoxy-5-oxo-2,3-dihydro-1H,5H-pyrrolizin-7a-ylmethyl]-benzamide, such as 4-[(2S,7aS)-2-acetylamino-6-(3,5-dichloro-phenyl)-7-methoxy-5-oxo-2,3-dihydro-1H,5H-pyrrolizin-7a-ylmethyl]-benzamide;

N-Benzyl-N-[7a-(4-cyano-benzyl)-6-(3,5-dichloro-phenyl)-7-methoxy-5-oxo-2,3,5,7a-tetrahydro-1H-pyrrolizin-2-yl]-acetamide, such as N-benzyl-N-[(2S,7aS)-7a-(4-cyano-benzyl)-6-(3,5-dichloro-phenyl)-7-methoxy-5-oxo-2,3,5,7a-tetrahydro-1H-pyrrolizin-2-yl]-acetamide;

N-[(7a-(4-Cyano-benzyl)-6-(3,5-dichloro-phenyl)-7-methoxy-5-oxo-2,3,5,7a-tetrahydro-1H-pyrrolizin-2-yl]-N-ethyl-acetamide, such as N-[(2S,7aS)-7a-(4-cyano-benzyl)-6-(3,5-dichloro-phenyl)-7-methoxy-5-oxo-2,3,5,7a-tetrahydro-1H-pyrrolizin-2-yl]-N-ethyl-acetamide;

1-[7a-(4-Cyano-benzyl)-6-(3,5-dichloro-phenyl)-7-methoxy-5-oxo-2,3,5,7a-tetrahydro-1H-pyrrolizin-2-yl]-1H-[1,2,3]triazole-4-carboxylic acid methyl ester, such as 1-[(2S,7aS)-7a-(4-cyano-benzyl)-6-(3,5-dichloro-phenyl)-7-methoxy-5-oxo-2,3,5,7a-tetrahydro-1H-pyrrolizin-2-yl]-1H-[1,2,3]triazole4-carboxylic acid methyl ester;

4-[6-(3,5-Dichloro-phenyl)-7-methoxy-5-oxo-2-(4-phenyl-[1,2,3]triazol-1-yl)-2,3-dihydro-1H,5H-pyrrolizin-7a-ylmethyl]-benzonitrile, such as 4-[(2S,7aS)-6-(3,5-dichloro-phenyl)-7-methoxy-5-oxo-2-(4-phenyl-[1,2,3]triazol-1-yl)-2,3-dihydro-1H,5H-pyrrolizin-7a-ylmethyl]-benzonitrile;

4-{6-(3,5-Dichloro-phenyl)-2-[5-iodo-4-(3-methyl-butyl)-[1,2,3]triazol-1-yl]-7-methoxy-5-oxo-2,3-dihydro-1H,5H-pyrrolizin-7a-ylmethyl}-benzonitrile, such as 4-{(2S,7aS)-6-(3,5-dichloro-phenyl)-2-[5-iodo-4-(3-methyl-butyl)-[1,2,3]triazol-1-yl]-7-methoxy-5-oxo-2,3-dihydro-1H,5H-pyrrolizin-7a-ylmethyl}-benzonitrile;

4-{(2S,7aS)-6-(3,5-Dichloro-phenyl)-7-methoxy-2-[4-(3-methyl-butyryl)-[1,2,3]triazol-1-yl]-5-oxo-2,3-dihydro-1H,5H-pyrrolizin-7a-ylmethyl}-benzonitrile, such as 4-{(2S,7aS)-6-(3,5-dichloro-phenyl)-7-methoxy-2-[4-(3-methyl-butyryl)-[1,2,3]triazol-1-yl]-5-oxo-2,3-dihydro-1H,5H-pyrrolizin-7a-ylmethyl}-benzonitrile;

4-{(2S,7aS)-6-(3,5-Dichloro-phenyl)-7-methoxy-2-[4-(3-methyl-butyl)-[1,2,3]triazol-1-yl]-5-oxo-2,3-dihydro-1H,5H-pyrrolizin-7a-ylmethyl}-benzonitrile, such as 4-{(2S,7aS)-6-(3,5-dichloro-phenyl)-7-methoxy-2-[4-(3-methyl-butyl)-[1,2,3]triazol-1-yl]-5-oxo-2,3-dihydro-1H,5H-pyrrolizin-7a-ylmethyl}-benzonitrile;

4-{6-(3,5-Dichloro-phenyl)-2-[5-iodo-4-(3-methyl-butyryl)-[1,2 ,3]triazol-1-yl]-7-methoxy-5-oxo-2,3-dihydro-1H,5H-pyrrolizin-7a-ylmethyl}-benzonitrile, such as 4-{(2S,7aS)-6-(3,5-dichloro-phenyl)-2-[5-iodo-4-(3-methyl-butyryl)-[1,2,3]triazol-1-yl]-7-methoxy-5-oxo-2,3-dihydro-1H,5H-pyrrolizin-7a-ylmethyl}-benzonitrile;

4-[6-(3,5-Dichloro-phenyl)-2-(5-iodo-4-trimethylsilanyl-[1,2,3]triazol-1-yl)-7-methoxy-5-oxo-2,3-dihydro-1H,5H-pyrrolizin-7a-ylmethyl]-benzonitrile, such as 4-[(2S,7aS)-6-(3,5-dichloro-phenyl)-2-(5-iodo-4-trimethylsilanyl-[1,2,3]triazol-1-yl)-7-methoxy-5-oxo-2,3-dihydro-1H,5H-pyrrolizin-7a-ylmethyl]-benzonitrile;

4-[6-(3,5-Dichloro-phenyl)-2-(5-iodo-[1,2,3]triazol-1-yl)-7-methoxy-5-oxo-2,3-dihydro-1H,5H-pyrrolizin-7a-ylmethyl]-benzonitrile, such as 4-[(2S,7aS)-6-(3,5-dichloro-phenyl)-2-(5-iodo-[1,2,3]triazol-1-yl)-7-methoxy-5-oxo-2,3-dihydro-1H,5H-pyrrolizin-7a-ylmethyl]-benzonitrile;

4-[6-(3,5-Dichloro-phenyl)-7-methoxy-5-oxo-2-(4-trimethylsilanyl-[1,2,3]triazol-1-yl)-2,3-dihydro-1H,5H-pyrrolizin-7a-ylmethyl]-benzonitrile, such as 4-[(2S,7aS)-6-(3,5-Dichloro-phenyl)-7-methoxy-5-oxo-2-(4-trimethylsilanyl-[1,2,3]triazol-1-yl)-2,3-dihydro-1H,5H-pyrrolizin-7a-ylmethyl]-benzonitrile;

4-[6-(3,5-Dichloro-phenyl)-7-methoxy-5-oxo-2-(4-pyridin-2-yl-[1,2,3]triazol-1-yl)-2,3-dihydro-1H,5H-pyrrolizin-7a-ylmethyl]-benzonitrile, such as 4-[(2S,7aS)-6-(3,5-dichloro-phenyl)-7-methoxy-5-oxo-2-(4-pyridin-2-yl-[1,2,3]triazol-1-yl)-2,3-dihydro-1H,5H-pyrrolizin-7a-ylmethyl]-benzonitrile;

4-[6-(3,5-Dichloro-phenyl)-7-methoxy-5-oxo-2-(4-pyridin-3-yl-[1,2,3]triazol-1-yl)-2,3-dihydro-1H,5H-pyrrolizin-7a-ylmethyl]-benzonitrile, such as 4-[(2S,7aS)-6-(3,5-dichloro-phenyl)-7-methoxy-5-oxo-2-(4-pyridin-3-yl-[1,2,3]triazol-1-yl)-2,3-dihydro-1H,5H-pyrrolizin-7a-ylmethyl]-benzonitrile;

4-[6-(3,5-Dichloro-phenyl)-2-(5-iodo-4-isobutyryl-[1,2,3]triazol-1-yl)-7-methoxy-5-oxo-2,3-dihydro-1H,5H-pyrrolizin-7a-ylmethyl]-benzonitrile, such as 4-[(2S,7aS)-6-(3,5-dichloro-phenyl)-2-(5-iodo-4-isobutyryl-[1,2,3]triazol-1-yl)-7-methoxy-5-oxo-2,3-dihydro-1H,5H-pyrrolizin-7a-ylmethyl]-benzonitrile;

4-[6-(3,5-Dichloro-phenyl)-2-(4-isobutyl-[1,2,3]triazol-1-yl)-7-methoxy-5-oxo-2,3-dihydro-1H,5H-pyrrolizin-7a-ylmethyl]-benzonitrile, such as 4-[(2S,7aS)-6-(3,5-dichloro-phenyl)-2-(4-isobutyl-[1,2,3]triazol-1-yl)-7-methoxy-5-oxo-2,3-dihydro-1H,5H-pyrrolizin-7a-ylmethyl]-benzonitrile;

4-[6-(3,5-Dichloro-phenyl)-2-(4-isobutyryl-[1,2,3]triazol-1-yl)-7-methoxy-5-oxo-2,3-dihydro-1H,5H-pyrrolizin-7a-ylmethyl]-benzonitrile, such as 4-[(2S,7aS)-6-(3,5-dichloro-phenyl)-2-(4-isobutyryl-[1,2,3]triazol-1-yl)-7-methoxy-5-oxo-2,3-dihydro-1H,5H-pyrrolizin-7a-ylmethyl]-benzonitrile;

4-[2-(4-Cyclopentyl-5-iodo-[1,2,3]triazol-1-yl)-6-(3,5-dichloro-phenyl)-7-methoxy-5-oxo-2,3-dihydro-1H,5H-pyrrolizin-7a-ylmethyl]-benzonitrile, such as 4-[(2S,7aS)-2-(4-Cyclopentyl-5-iodo-[1,2, 3]triazol-1-yl)-6-(3,5-dichloro-phenyl)-7-methoxy-5-oxo-2,3-dihydro-1H,5H-pyrrolizin-7a-ylmethyl]-benzonitrile;

4-[2-(4-Cyclopentyl-[1,2,3]triazol-1-yl)-6-(3,5-dichloro-phenyl)-7-methoxy-5-oxo-2,3-dihydro-1H,5H-pyrrolizin-7a-ylmethyl]-benzonitrile, such as 4-[(2S,7aS)-2-(4-cyclopentyl-[1,2,3]triazol-1-yl)-6-(3,5-dichloro-phenyl)-7-methoxy-5-oxo-2,3-dihydro-1H,5H-pyrrolizin-7a-ylmethyl]-benzonitrile;

4-[6-(3,5-Dichloro-phenyl)-2-(5-iodo-4-tridecyl-[1,2,3]triazol-1-yl)-7-methoxy-5-oxo-2,3-dihydro-1H,5H-pyrrolizin-7a-ylmethyl]-benzonitrile, such as 4-[(2S,7aS)-6-(3,5-dichloro-phenyl)-2-(5-iodo-4-tridecyl-[1,2,3]triazol-1-yl)-7-methoxy-5-oxo-2,3-dihydro-1H-5H-pyrrolizin-7a-ylmethyl]-benzonitrile;

4-[6-(3,5-Dichloro-phenyl)-7-methoxy-5-oxo-2-(4-tridecyl-[1,2,3]triazol-1-yl)-2,3-dihydro-1H,5H-pyrrolizin-7a-ylmethyl]-benzonitrile, such as 4-[(2S,7aS)-6-(3,5-dichloro-phenyl)-7-methoxy-5-oxo-2-(4-tridecyl-[1,2,3]triazol-1-yl)-2,3-dihydro-1H,5H-pyrrolizin-7a-ylmethyl]-benzonitrile;

4-[6-(3,5-Dichloro-phenyl)-2-(5-iodo-4-tridecanoyl-[1,2,3]triazol-1-yl)-7-methoxy-5-oxo-2,3-dihydro-1H,5H-pyrrolizin-7a-ylmethyl]-benzonitrile, such as 4-[(2S,7aS)-6-(3,5-dichloro-phenyl)-2-(5-iodo-4-tridecanoyl-[1,2,3]triazol-1-yl)-7-methoxy-5-oxo-2,3-dihydro-1H,5H-pyrrolizin-7a-ylmethyl]-benzonitrile, AND

4-[Azido-6-(3,5-dichloro-phenyl)-7-methoxy-5-oxo-2,3-dihydro-1H,5H-pyrrolizin-7a-ylmethyl]-benzonitrile, such as 4-[(2S,7aS)-2-Azido-6-(3,5-dichloro-phenyl)-7-methoxy-5-oxo-2,3-dihydro-1H,5H-pyrrolizin-7a-ylmethyl]-benzonitrile;

such as N-[(2S,7aS)-7a-(4-cyano-benzyl)-6-(3,5-dichloro-phenyl)-7-methoxy-5-oxo-2,3,5,7a-tetrahydro-1H-pyrrolizin-2-yl]-acetamide, e.g. compounds such as disclosed in TABLE 1 and TABLE 2 in the Example part herein with the exception of Examples 33, 34, 38, 39, 44 and 45 which Examples refer to compounds which are useful as intermediates in the preparation of compounds of formula I,

Compounds of formula I provided by the present invention are hereinafter designated as “compound(s) of (according to) the present invention”.

A compound of the present invention includes a compound in any form, e.g. in free form, in the form of a salt, in the form of a solvate and in the form of a salt and a solvate.

In another aspect the present invention provides a compound of the present invention in the form of a salt.

Such salts include preferably pharmaceutically acceptable salts, although pharmaceutically unacceptable salts are included, e.g. for preparation/isolation/purification purposes.

A salt of a compound of the present invention includes a metal salt or an acid addition salt. A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention and an intermediate of the present invention, may exist in the form of isomers and mixtures thereof; e.g. optical isomers, diastereoisomers, cis/trans conformers. A compound of the present invention and an intermediate of the present invention may e.g. contain asymmetric carbon atoms and may thus exist in the form of enatiomers or diastereoisomers and mixtures thereof, e.g. racemates. A compound of the present invention and an intermediate of the present invention may be present in the (R)-, (S)- or (R,S)-configuration preferably in the (R)- or (S)-configuration regarding substituents in specified positions in a compound of the present invention. For example, the carbon atoms in position 5 of ring A in a group of formula A1, A2, A3, A4 or A5, or the methylene group to which R₄ is attached in the ring which is not ring A in a compound of formula I, both are asymmetric carbon atoms and a compound of the present invention may be present in the (R)-, (S)- or (R,S)-configuration preferably in the (R)- or (S)-configuration regarding each of the substituents at such asymmetric carbon atoms in a compound of the present invention, e.g. a compound of the present invention may be present in the (R)-, (S)- or (R,S)-configuration, preferably in the (R)- or (S)-configuration regarding R₃, or R₄ respectively, attached in the positions as specified above.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of the present invention, where tautomers can exist.

In another aspect the present invention provides a process for the production of a compound of formula I,

a. for the preparation of a compound of formula I wherein ring A is a group of formula A3 or A5 comprising the steps

a1. reacting a compound of formula

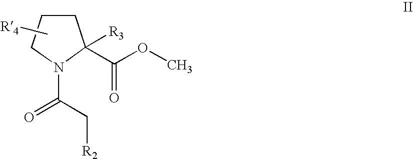

wherein R₂, R₃ are as defined above and and R′₄ has the meaning of R₄ as defined above and additionally R′₄ is hydroxy, oxo or acyloxy, e.g. methylcarbonyloxy, with potassium hexamethyl silazane, e.g. in organic solvent,

a2. isolating a compound of formula I, wherein R₂, R₃ and R′₄ are as defined above, and wherein R₆ is hydroxy from the reaction mixture, and optionally

a3 further reacting a compound obtained in step a2. to obtain a compound of formula I, wherein ring A is a group of formula A1, A2, A3, A4 or A5, wherein R₅, R₆, R₂ and R₃ are as defined above and R′₄ has the meaning of R₄ as defined above.

A compound of formula I wherein ring A is a group of formula A1, A2 or A4 may be obtained from a compound obtained in step a2. by further reacting with a reactive derivative, e.g. a halogenide of

alkyl, e.g. (C₁₋₁₈)alkyl, such as (C₁₋₆)alkyl; e.g. (C₁₋₄)alkyl; alkenyl, e.g. (C₂₋₁₈)alkenyl, such as (C₂₋₆)alkenyl, alkynyl, (C₂₋₁₈)alkynyl, such as (C₂₋₄)alkynyl, or

alkyl, alkenyl or alkynyl substituted by
alkyoxy, e.g. (C₁₋₄)alkoxy,
a silyl or silyloxy group, such as trialkylsilyl, e.g. tri(C₁₋₆)alkylsilyl, such as trimethylsilyl or (tert. butyl)(dimethyl)silyloxy,
cycloalkyl, e.g. (C₃₋₁₈)cycloalkyl,
aryl, e.g. (C₆₋₁₈)aryl, such as (C₆₋₁₂)aryl, or
heterocyclyl.

A compound of formula I wherein R₆ is other than hydroxy may be obtained in step 3a. by further treating the hydroxy group of R₆ as obtained in step a2. by alkylating, acylating, sulfonylating, e.g. according, e.g. analogously, to a method as conventional; in the course of that reaction also a compound of formula 1, wherein ring A is a group of formula A1, A2 or A4 may be obtained.

If R′₄ is other than R₄ in a compound of formula II, step a3. e.g. may be carried out as follows:

E.g. if R′₄ is acyloxy, such as methylcarbonyloxy, the acyl group may be split off, e.g. by a method for ester saponification, to obtain a hydroxy group.

E.g. if R′₄ is oxo, said oxo group may be reduced to a hydroxy group.

E.g. if R′₄ is hydroxy, said hydroxy group may be further reacted, e.g. silylated or replaced via substitution with an amine, N₃ or heterocyclyl to obtain a compound of formula I wherein R₄ is as defined above.

A compound of formula I thus obtained may be converted into another compound of formula I, e.g. or a compound of formula I obtained in free form may be converted into a salt of a compound of formula I and vice versa.

In another aspect the present invention provides a compound selected from the group consisting of

1,6-Dihydroxy-7a-methyl-2-phenyl-5,6,7,7a-tetrahydro-pyrrolizin-3-one, such as (6R,7aS)-1,6-Dihydroxy-7a-methyl-2-phenyl-5,6,7,7a-tetrahydro-pyrrolizin-3, 7a-(4-Bromo-benzyl)-2-(3,5-dichloro-phenyl)-1,6-dihydroxy-5,6,7,7a-tetrahydro-pyrrolizin-3-one, such as (6R,7aS)-7a-(4-Bromo-benzyl)-2-(3,5-dichloro-phenyl)-1,6-dihydroxy-5,6,7,7a-tetrahydro-pyrrolizin-3-one;

A compound of formula

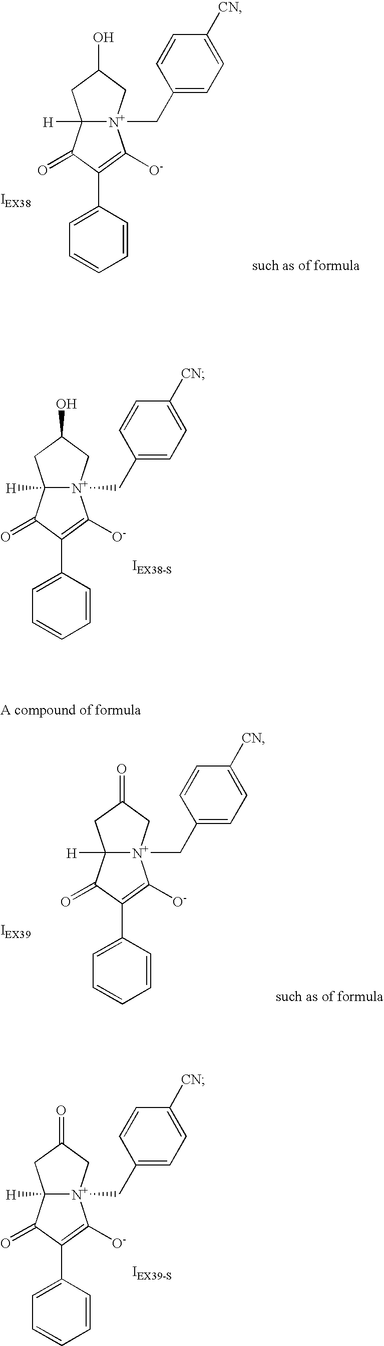

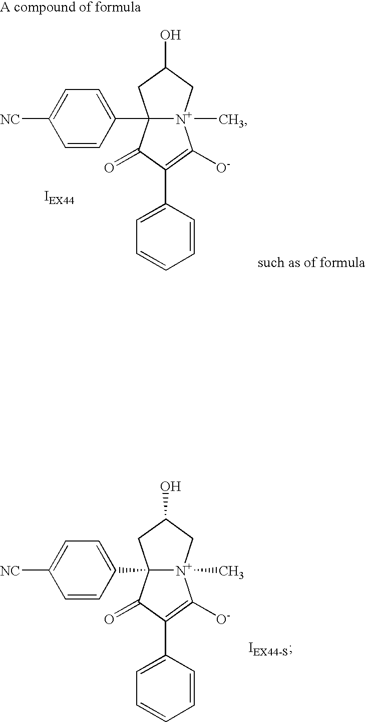

and

Acetic acid 7a-(4-cyano-benzyl)-6-(3,5-dichloro-phenyl)-7-methoxy-5-oxo-2,3,5,7a-tetrahydro-1H-pyrrolizin-2-yl ester, such as acetic acid (S)-7a-(4-cyano-benzyl)-6-(3,5-dichloro-phenyl)-7-methoxy-5-oxo-2,3,5,7a-tetrahydro-1H-pyrrolizin-2-yl ester;

e.g. which compounds are useful as intermediates for the preparation of compounds of formula I; and which compounds are herein also designated as “intermediates of (according to) the present invention”.

A compound of formula II may be e.g. obtained by reacting a compound of formula

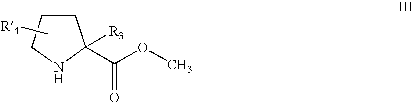

wherein R₃ and R′₄ are as defined above, with an activated form of a compound of formula

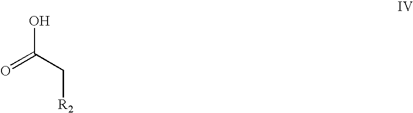

e.g. a corresponding acid halogenide, such as an acid chloride, wherein R₂ is as defined above in organic solvent in the presence of base, e.g. aqueous potassium hydrogen carbonate.

A compound of formula III may be e.g. obtained by reacting a compound of formula

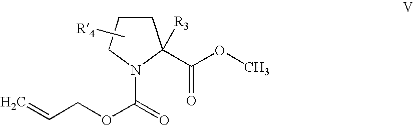

wherein R₃ and R₄ are as defined above, with tetrakis-(triphenylphosphine)-palladium in organic solvent.

A compound of formula V may be e.g. obtained, if R′₄ in a compound of formula I is other than N₃ or oxo, by reacting a compound of formula

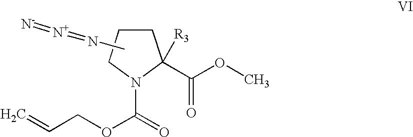

wherein R₃ is as defined above, with zinc powder in glacial acetic acid, to obtain a compound of formula

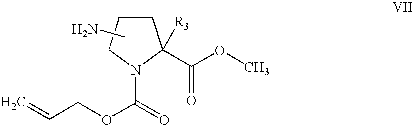

and optionally further reacting the amine group obtained, to obtain a compound of formula V, wherein R₃ and R′₄ are as defined above, with the exception of R′₄ is oxo.

A compound of formula VI, wherein R₃ is other than hydrogen, may be e.g. obtained by treating a compound of formula

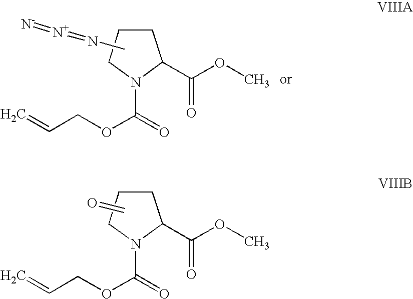

with a strong base, e.g. lithium-bis-trimetylsilylamide with lithium-bis-trimetylsilylamide in organic solvent and treating the mixture obtained with a compound of formula

R₃—Hal IX

wherein R₃ is as defined above with the exception of hydrogen, and Hal is halogen, such as Br.

In any intermediate of formulae II, III, IV, V, VI or VII, VIIIA, VIIIB and IX, functional groups, if present, optionally may be in protected form or in the form of a salt, if a salt-forming group is present. Protecting groups, optionally present, may be removed at an appropriate stage, e.g. according, e.g. analogously, to a method as conventional

Any compound described herein, e.g. a compound of the present invention and intermediates (starting materials) of formulae II, III, IV, V, VI, VII, VIIIA, VIIIB or IX may be prepared as appropriate, e.g. according, e.g. analogously, to a method as conventional, e.g. or as specified herein.

The compounds of the present invention exhibit valuable pharmacological properties, e.g. by mediating, such as inhibiting the activity of LFA-1 interactions with its ligands, e.g. inhibiting the activity of LFA-1/ICAM-1, LFA-1/ICAM-2, LFA-1/ICAM-3 and/or LFA-1/JAM-1 interactions, e.g. LFA-1/ICAM-1 interaction, and thus mediating, e.g. inhibiting inflammation, e.g. as indicated in vitro and in vivo TEST SYSTEMS herein. The compounds of the present invention are therefore indicated for therapy.

A. In vitro TEST SYSTEM (Cell Free Assay)

The assay determines the binding of soluble human ICAM-1 to immobilized human LFA-1. LFA-1 is purified from JY cells, a human lymphoblastoid B cell-line, by immunoaffinity chromatography analogously as described by Dustin et al., J. Immunol. 148, 2654-2663, 1992. ICAM-1 mouse CK fusion protein (ICAM-1) is produced using the baculovirus system as described by Weitz-Schmidt et al., Anal. Biochem. 238,184-190, 1996.

Purified LFA-1 is diluted 1:20 in phosphate buffered saline (PBS) containing 2 mM MgCl₂, pH 7.4 and coated onto microtiter plates (Nunc) at 37° for 3 hours. Plates are blocked with 1% heat-treated bovine serum albumin in PBS for 2 hours at 370 followed by a washing step using PBS, 2 mM MgCl₂, 1% fetal calf serum, pH 7.4 (assay buffer). Compounds of the present invention (10 mM solution in DMSO) are diluted in assay buffer and added to the plates. Biotinylated recombinant ICAM-1 in assay buffer (6 μg/ml) is added and allowed to bind at 370 for one hour. After incubation, wells are washed with assay buffer. Streptavidin-peroxidase diluted 1:5000 in assay buffer is added and incubated for 45 min at 37°. Plates are washed with assay buffer and 2,2′-azino-bis(3-ethylbenzothiazoline-6-sulfonic acid) diammonium salt substrate solution is added to each well. The reaction is stopped after 20 minutes and bound ICAM-1 is determined by measuring the optical density at 405 nm in a microplate reader.

In this assay the compounds of the present invention exhibit activity, e.g. the compounds of the present invention inhibit adhesion of LFA-1 to ICAM.

B. In vivo TEST SYSTEM Allergic Contact Dermatitis (ACD)

Groups of 8 female NMRI mice are sensitized on the shaved abdomen with 50 μl of oxazolone (2% in acetone) and challenged with 10 μl of 0.2% oxazolone on the inner surface of the right ear 7 days later. The unchallenged left ears serve as normal controls and dermatitis is evaluated from the individual differences in auricular weights, which are taken as a measure of inflammatory swelling 24 hours after the challenge. The test groups are treated with the test compounds orally (2 hours after challenge), the controls are treated similarly with the vehicles alone. For oral administration the compounds are administered in an oil in H₂O emulsion. Dermatitis is evaluated in test- and control groups. The animals are killed and both ears are cut off and weighed. Inhibitory activity of the test compounds is calculated from the differences in right and left ears (internal controls) in mice treated with test compounds compared with animals treated with the vehicle only. The data of the test- and the vehicle-treated control groups are statistically analyzed by ANOVA followed by Dunnet T-test (normal distribution or data) or by H and U-test, respectively. The compounds of the present invention inhibit the elicitation phase of allergic contact dermatitis based on the differences in auricular weights.

The compounds of the present invention are therefore indicated for use in the treatment or prevention of disorders, including diseases, mediated by interactions of LFA-1 with its ligands involved in cell adhesion, migration and activation. The compounds of the present invention may be preferably useful for treatment or prevention of inflammatory conditions, allergic diseases, autoimmune diseases, transplant rejection, antiproliferative or infective diseases.

Disorders mediated by interactions of LFA-1 with its ligands involved in cell adhesion, migration and activation have been reported to include e.g.

Disorders Associated With Inflammation

e.g. including (chronic) inflammatory disorders, disorders related with the inflammation of the bronchi, e.g. including bronchitis, cervix, e.g. including cervicitis, conjunctiva, e.g. conjunctivitis, esophagus, e.g. esophagitis, heart muscle, e.g. myocarditis, rectum, e.g. proctitis, sclera, e.g. scleritis, gums, involving bone, pulmonary inflammation (alveolitis), airways, e.g. asthma, such as bronchial asthma, acute respiratory distress syndrome (ARDS), inflammatory skin disorders such as contact hypersensitivity, (allergic) contact dermatitis, atopic dermatitis; fibrotic disease (e.g., pulmonary fibrosis), encephalitis, inflammatory osteolysis,

Disorders Associated With Conditions of the Immune System,

immune, such as autoimmune disorders e.g. including Graves' disease, Hashimoto's disease (chronic thyroiditis), multiple sclerosis, rheumatoid arthritis, arthritis, gout, osteoarthritis, scieroderma, lupus syndromes, systemic lupus erytomatosis, Sjoegren's syndrome, psoriasis, inflammatory bowel disease, including Crohn's disease, colitis, e.g. ulcerative colitis; sepsis, septic shock, autoimmune hemolytic anemia (AHA), autoantibody triggered urticaria, pemphigus, nephritis, glomerulonephritis, Goodpastur syndrom, ankylosing spondylitis, Reiter's syndrome, polymyositis, dermatomyositis, cytokine-mediated toxicity, interleukin-2 toxicity, alopecia, e.g. alopecia areata, hair growth, uveitis, lichen planus, bullous pemphigoid, myasthenia gravis, type I diabetes mellitus, immune-mediated infertility such as premature ovarian failure, polyglandular failure, hypothyroidism, pemphigus vulgaris, pemphigus I-oliaceus, paraneoplastic pemphigus, autoimmune hepatitis including that associated with hepatitis B virus (HBV) and hepatitis C virus (HCV), Addison's disease, autoimmune skin diseases, such as psoriasis, dermatitis herpetiformis, epidermolysis bullosa, linear IgA bullous dermatosis, epidermolysis bullosa acquisita, chronic bullous disease of childhood, pernicious anemia, hemolytic anemia, vitiligo, type I, type II and type III autoimmune polyglandular syndromes, Autoimmune Hypoparathyroidism, Autoimmune Hypophysitis, Autoimmune Oophoritis, Autoimmune Orchitis, pemphigoid gestationis, cicatricial pemphigoid, mixed essential cryoglobulinemia, immune thrombocytopenic purpura, Goodpasture's syndrome, autoimmune neutropenia, Eaton-Lambert myasthenic syndrome, stiff-man syndrome, encephalomyelitis, acute disseminated encephalomyelitis, Guillain-Barre syndrome, cerebellar degeneration, retinopathy, primary biliary sclerosis, sclerosing cholangitis autoimmune hepatitis, gluten-sensitive enteropathy, reactive arthritides, polymyositis/dermatomyositis, mixed connective tissue disease, Bechet's syndrome, polyarteritis nodosa allergic anguitis and granulomatosis (Churg-Strauss disease), polyangiitis overlap syndrome (hypersensitivity) vasculitis, Wegener's granulomatosis, temporal arteritis Kawasaki's disease, sarcoidosis, cryopathies, Celiac disease,

Disorders Associated With Cytokine-Mediated Toxicity,

e.g. including interleukin-2 toxicity,

Disorders Associated With the Bone,

e.g. including osteoporosis, osteoarthritis,

Disorders Associated With the Brain and the Nerves,

neurodegenerative disorders, e.g. including disorders of the central nervous system as well as disorders of the peripheral nervous system, e.g. CNS disorders including central nervous infections, brain injuries, cerebrovascular disorders and their consequences, Parkinson's disease, corticobasal degeneration, motor neuron disease, dementia including ALS, multiple sclerosis, traumatic disorders, including trauma and inflammatory consequences of trauma, traumatic brain injury, stroke, post-stroke, post-traumatic brain injury,

small-vessel cerebrovascular disease, eating disorders; further dementias, e.g. including Alzheimer's disease, vascular dementia, dementia with Lewy-bodies, frontotemporal dementia and Parkinsonism linked to chromosome 17, frontotemporal dementias, including Pick's disease, progressive nuclear palsy, corticobasal degeneration, Huntington's disease, thalamic degeneration, Creutzfeld Jakob dementia, HIV dementia, schizophrenia with dementia, Korsakoff's psychosis,

cognitive-related disorders, such as mild cognitive impairment, age associated memory impairment, age-related cognitive decline, vascular cognitive impairment, attention deficit disorders, attention deficit hyperactivity disorders, and memory disturbances in children with learning disabilities; conditions associated with the hypothalamic-pituitary-adrenal axis, neuronal disorders, e.g, including neuronal migration disorders, hypotonia (reduced muscle tone), muscle weakness, seizures, developmental delay (physical or mental development difficulty), mental retardation, growth failure, feeding difficulties, lymphedema, microcephaly, symptoms affecting the head and the brain, motor dysfunction;

Disorders Associated With the Eye,

e.g. including uveoritinitis, vitreoretinopathy, corneal disease, iritis, iridocyclitis, cateracts, uveitis, diabetic retinopathy, retinitis pigmentosa, conjunctivits, keratitis,

Disorders Associated With the Gastrointestinal Tract

e.g. including colitis, inflammatory bowel disease, colitis, Crohn's disease, ulcerative colitis, peptic ulceration, gastritis, esophagitis,

Disorders Associated With the Heart and Vascular Conditions

e.g. including cardiovascular disorders, e.g. including cardiac failure, cardiac infarction, cardiac hypertrophy, heart failure, e.g. including all forms of heart pumping failures such as high-output and low-output, acute and chronic, right sided or left-sided, systolic or diastolic, independent of the underlying cause; myocardial infarction (MI), MI prophylaxis (primary and secondary prevention), acute treatment of MI, prevention of complications; heart disorders, proliferative vascular disorders, vasculitides, polyarteritis nodosa, inflammatory consequences of ischemia, ischemic heart disease, myocardial infarction, stroke, peripheral vascular disease, pulmonary hypertension,

ischemic disorders, e.g. including myocardial ischemia, e.g. stable angina, unstable angina, angina pectoris, bronchitis; asymptomatic arrhythmias such as all forms of atrial and ventricular tachyarrhythmias, atrial tachycardia, atrial flutter, atrial fibrillation, atrio-ventricular reentrant tachycardia, preexitation syndrome, ventricular tachycardia, ventricular flutter, ventricular fibrillation, bradycardic forms of arrhythmias; arrhythmia, chronic obstructive pulmonary disease,

hypertension, such as systolic or diastolic high blood pressure, e.g essential and secondary hypertension, e.g. including hypertensive vascular disorders, such as primary as well as all kinds of secondary arterial hypertension, renal, endocrine, neurogenic and others;

peripheral vascular disorders in which arterial and/or venous flow is reduced resulting in an imbalance between blood supply and tissue oxygen demand, e.g. including artherosclerosis, chronic peripheral arterial occlusive disease (PAOD), acute arterial thrombosis and embolism, inflammatory vascular disorders, Raynaud's phenomenon and venous disorders; atherosclerosis, a disease in which the vessel wall is remodeled, e.g. including accumulation of cells, both smooth muscle cells and monocytelmacrophage inflammatory cells, in the intima of the vessel wall; hypotension,

Disorders Associated With the Liver and the Kidneys,

e.g. including renal disorders, kidney disorders, e.g. acute kidney failure, acute renal disease, liver disorders, e.g. cirrhosis, hepatitis, liver failure, cholestasis, acute/chronic hepatitis, sclerosing cholangitis, primary biliary cirrhosis, acute/chronic interstitial/glomerulonephritis, granulomatous diseases,

Disorders Associated With Stomach or Pancreas Conditions

e.g. including stomach disorders, e.g. gastric ulcer, gastrointestinal ulcer, pancreatic disorders pancreatic fatigue,

Disorders Associated With the Respiratory Tract and Lung

e.g. including pulmonary disorders, chronic pulmonary disease, acute (adult) respiratory distress syndrome (ARDS), asthma, asthma bronchitis, bronchiectasis, diffuse interstitial lung disorders, pneumoconioses, fibrosing aveolitis, lung fibrosis,

Disorders Associated With Skin and Connective Tissue Conditions

e.g. including eczema, atopic dermatitis, (allergic) contact dermatitis, psoriasis, acne, dermatomyositis, Sjörgen's syndrome, Churg-Struass syndrome, sunburn, skin cancer, wound healing, urticaria, toxic epidermal necrolysis, age related skin conditions, cellulite,

Disorders Associated With Allergic Conditions,

e.g. including delayed-type hypersensitivity, allergic conjunctivitis, drug allergies, rhinitis, allergic rhinitis, vasculitis, contact dermatits;

Disorders Associated With Angiogenesis,

e.g. including insufficient ability to recruit blood supply, disorders characterised by modified angiogenesis, tumor associated angiogenesis,

Disorders Associated With Cancer and Cell Overproliferation,

e.g. including premalignant conditions, hyperproliferative disorders, cancers whether primary or metastatic, cervical and metastatic cancer, cancer originating from uncontrolled cellular proliferation, solid tumors, such as such as described in WO02066019, including nonsmall cell lung cancer, cervical cancer; tumor growth, lymphoma, B-cell or T-cell lymphoma, benign tumors, benign dysproliferative disorders, renal carcinoma, esophageal cancer, stomach cancer, renal carcinoma, bladder cancer, breast cancer, colon cancer, lung cancer, melanoma, nasopharyngeal cancer, osteocarcinoma, ovarian cancer, uterine cancer; prostate cancer, skin cancer, leukemia, tumor neovascularization, angiomas, myelodysplastic disorders, unresponsiveness to normal death-inducing signals (immortalization), increased cellular motility and invasiveness, genetic instability, dysregulated gene expression, (neuro)endocrine cancer (carcinoids), blood cancer, lymphocytic leukemias, neuroblastoma; soft tissue cancer, prevention of metastasis,

Disorders Associated With Diabetic Conditions,

e.g. including diabetes (type I diabetes, type II diabetes), diabetic retinopathy, insulin-dependent diabetes, diabetes mellitus, gestational diabetes), insulin hyposecretion, obesity;

Disorders Associated With Endometriosis, Testicular Dysfunctions,

Disorders Associated With Infectious Disorders, e.g. With Chronic Infectious Conditions,

e.g. including bacterial disorders, otitis media, Lyme disease, thryoditis, viral disorders, parasitic disorders, fungal disorders, malaria, e.g. malaria anemia, sepsis, severe sepsis, septic shock, e.g. endotoxin-induced septic shock, exotoxin-induced toxic shock, infective (true septic) shock, septic shock caused by Gram-negative bacteria, pelvic inflammatory disease, AIDS, enteritis, pneumonia; meningitis, encephalitis,

Disorders Associated With Myasthenia Gravis,

Disorders Associated With Nephritis,

e.g. including glomerulonephritis, interstitial nephritis, Wegener's granulomatosis, fibrosis,

Disorders Associated With Pain,

e.g. associated with CNS disorders, such as multiple sclerosis, spinal cord injury, sciatica, failed back surgery syndrome, traumatic brain injury, epilepsy, Parkinson's disease, post-stroke, and vascular lesions in the brain and spinal cord (e.g., infarct, hemorrhage, vascular malformation);

non-central neuropathic pain, e.g. including that associated with post mastectomy pain, phantom feeling, reflex sympathetic dystrophy (RSD), trigeminal neuralgia, radioculopathy, post-surgical pain, HIV/AIDS related pain, cancer pain, metabolic neuropathies (e.g., diabetic neuropathy, vasculitic neuropathy secondary to connective tissue disease), paraneoplastic polyneuropathy associated, for example, with carcinoma of lung, or leukemia, or lymphoma, or carcinoma of prostate, colon or stomach, trigeminal neuralgia, cranial neuralgias, and post-herpetic neuralgia;

pain associated with peripheral nerve damage, central pain (i.e. due to cerebral ischemia) and various chronic pain i.e., lumbago, back pain (low back pain), inflammatory and/or rheumatic pain; headache pain (for example, migraine with aura, migraine without aura, and other migraine disorders), episodic and chronic tension-type headache, tension-type like headache, cluster headache, and chronic paroxysmal hemicrania;

visceral pain such as pancreatits, intestinal cystitis, dysmenorrhea, irritable Bowel syndrome, Crohn's disease, biliary colic, ureteral colic, myocardial infarction and pain syndromes of the pelvic cavity, e.g., vulvodynia, orchialgia, urethral syndrome 15 and protatodynia;

acute pain, for example postoperative pain, and pain after trauma;

Disorders Associated With Rheumatic Disorders,

e.g. including arthritis, rheumatoid arthritis, osteoarthritis, psoriatic arthritis, crystal arthropathies, gout, pseudogout, calcium pyrophosphate deposition disease, lupus syndromes, systemic lupus erythematosus, sclerosis, sclerodema, multiple sclerosis, artherosclerosis, arteriosclerosis, spondyloarthropathies, systemic sclerosis, reactive arthritis, Reiter's syndrome, ankylosing spondylitis, polymyositis,

Disorders Associated With Sarcoidosis,

Disorders Associated With Transplantation,

e.g. including transplant rejection crisis and other disorders following transplantation, such as organ or tissue (xeno)transplant rejection, e.g. for the treatment of recipients of e.g. heart, lung, combined heart-lung, liver, kidney, pancreatic, skin, corneal transplants, graft versus host disease, such as following bone marrow transplantation, ischemic reperfusion injury.

The compound of the present invention are preferably useful for treatment of disorders associated with conditions of the immune system, inflammation and transplantation; e.g. including psoriasis, rheumatoid arthritis, inflammatory bowel diseases (Crohn's disease, ulcerative colitis), (systemic) lupus erythematosus, multiple sclerosis, Sjoegren's syndrom, rejection after transplantation and graft vs. host disease and inflammatory skin diseases, e.g. dermatitis, such as atopic dermatitis, e.g. allergic contact dermatitis.

In one embodiment the compounds of the present invention are useful in the treatment of autoimmune diseases, e.g. rheumatoid arthritis, psoriasis, inflammatory bowel disease, (systemic) lupus erythematosus, multiple sclerosis or of inflammatory (skin) diseases, e.g. dermatitis;

more preferably in the treatment of inflammatory bowel disease, rheumatoid arthritis or dermatitis.

In another aspect the present invention provides
a compound of the present invention for use as a pharmaceutical,
the use of a compound of the present invention as a pharmaceutical,

e.g. for the treatment of disorders mediated by interactions of LFA-1 with its ligands involved in cell adhesion, migration and activation.

For pharmaceutical use one or more compounds of the present invention may be used, e.g. one, or a combination of two or more compounds of the present invention, preferably one compound of the present invention is used.

A compound of the present invention may be used as a pharmaceutical in the form of a pharmaceutical composition.

In another aspect the present invention provides a pharmaceutical composition comprising a compound of the present invention in association with at least one pharmaceutically acceptable excipient, e.g. appropriate carrier and/or diluent, e.g. including fillers, binders, disintegrants, flow conditioners, lubricants, sugars or sweeteners, fragrances, preservatives, stabilizers, wetting agents and/or emulsifiers, solubilizers, salts for regulating osmotic pressure and/or buffers.

In another aspect the present invention provides
a pharmaceutical composition of the present invention for use of treating disorders which are mediated by interactions of LFA-1 with its ligands involved in cell adhesion, migration and activation;
the use of a pharmaceutical composition of the present invention for treating disorders which are mediated by interactions of LFA-1 with its ligands involved in cell adhesion, migration and activation.

In a further aspect the present invention provides a method of treating disorders which are mediated by interactions of LFA-1 with its ligands involved in cell adhesion, migration and activation, which treatment comprises administering to a subject in need of such treatment an effective amount of a compound of the present invention; e.g. in the form of a pharmaceutical composition.

In another aspect the present invention provides

A compound of the present invention for the manufacture of a medicament, OR

The use of a compound of the present invention for the manufacture of a medicament, e.g. a pharmaceutical composition,

for the treatment of disorders, which are mediated by interactions of LFA-1 with its ligands involved in cell adhesion, migration and activation.

Treatment of disorders (diseases) as used herein includes treatment and prophylaxis (prevention).

For such treatment, the appropriate dosage will, of course, vary depending upon, for example, the chemical nature and the pharmacokinetic data of a compound of the present invention used, the individual host, the mode of administration and the nature and severity of the conditions being treated. However, in general, for satisfactory results in larger mammals, for example humans, an indicated daily dosage includes a range
from about 0.0001 g to about 1.5 g, such as 0.001 g to 1.5 g;
from about 0.001 mg/kg body weight to about 20 mg/kg body weight, such as 0.01 mg/kg body weight to 20 mg/kg body weight,

for example administered in divided doses up to four times a day.

A compound of the present invention may be administered to larger mammals, for example humans, by similar modes of administration than conventionally used with other mediators, e.g. low molecular weight inhibitors, of activity of LFA-1 with its ligands involved in cell adhesion, migration and activation.

A compound of the present invention may be administered by any route, e.g. by any conventional route, for example enterally, e.g. including nasal, buccal, rectal, oral, administration; parenterally, e.g. including intravenous, intraarterial, intramuscular, intracardiac, subcutanous, intraosseous infusion, transdermal (diffusion through the intact skin), transmucosal (diffusion through a mucous membrane), inhalational administration; topically; e.g. including epicutaneous, intranasal, intratracheal administration; intraperitoneal (infusion or injection into the peritoneal cavity); epidural (peridural) (injection or infusion into the epidural space); intrathecal (injection or infusion into the cerebrospinal fluid); intravitreal (administration via the eye); or via medical devices, e.g. for local delivery, e.g. stents, e.g. in form of coated or uncoated tablets, capsules, (injectable) solutions, infusion solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

For topical use, e.g. including administration to the eye, satisfactory results may be obtained with local administration of a 0.5-10%, such as 1-3% concentration of active substance several times daily, e.g. 2 to 5 times daily.

The compounds of the present invention may be administered in the form of a pharmaceutically acceptable salt, or in free form; optionally in the form of a solvate. A compound of the present invention in the form of a salt and/or in the form of a solvate exhibit the same order of activity as a compound of the present invention in free form.

A compound of the present invention may be used for any method or use as described herein alone or in combination with one or more, at least one, other, second drug substance.

In another aspect the present invention provides
A combination of a compound of the present invention with at least one second drug substance;
A pharmaceutical combination comprising a compound of the present invention in combination with at least one second drug substance;
A pharmaceutical composition comprising a compound of the present invention in combination with at least one second drug substance and one or more pharmaceutically acceptable excipient(s);
A compound of the present invention in combination with at least one second drug substance, e.g. in the form of a pharmaceutical combination or composition, for use in any method as defined herein, e.g.
A combination, a pharmaceutical combination or a pharmaceutical composition, comprising a compound of the present invention and at least one second drug substance for use as a pharmaceutical;
The use as a pharmaceutical of a compound of the present invention in combination with at least one second drug substance, e.g. in the form of a pharmaceutical combination or composistion;
The use of a compound of the present invention for the manufacture of a medicament for use in combination with a second drug substance;
A method for treating of disorders, which are mediated by interactions of LFA-1 with its ligands involved in cell adhesion, migration and activation, in a subject in need thereof, comprising co-administering, concomitantly or in sequence, a therapeutically effective amount of a compound of the present invention and at least one second drug substance, e.g. in the form of a pharmaceutical combination or composition;
A compound of the present invention in combination with at least one second drug substance, e.g. in the form of a pharmaceutical combination or composition, for use in the preparation of a medicament for the treatment of disorders which are mediated by interactions of LFA-1 with its ligands involved in cell adhesion, migration and activation.

Combinations include fixed combinations, in which a compound of the present invention and at least one second drug substance are in the same formulation; kits, in which a compound of the present invention and at least one second drug substance in separate formulations are provided in the same package, e.g. with instruction for co-administration; and free combinations in which a compound of the present invention and at least one second drug substance are packaged separately, but instruction for concomitant or sequential administration are given.

In another aspect the present invention provides
A pharmaceutical package comprising a first drug substance which is a compound of the present invention and at least one second drug substance, beside instructions for combined administration;
A pharmaceutical package comprising a compound of the present invention beside instructions for combined administration with at least one second drug substance;
A pharmaceutical package comprising at least one second drug substance beside instructions for combined administration with a compound of the present invention.

Treatment with combinations according to the present invention may provide improvements compared with single treatment.

In another aspect the present invention provides
A pharmaceutical combination comprising an amount of a compound of the present invention and an amount of a second drug substance, wherein the amounts are appropriate to produce a synergistic therapeutic effect;
A method for improving the therapeutic utility of a compound of the present invention comprising co-administering, e.g. concomitantly or in sequence, of a therapeutically effective amount of a compound of the present invention and a second drug substance.
A method for improving the therapeutic utility of a second drug substance comprising co-administering, e.g. concomitantly or in sequence, of a therapeutically effective amount of a compound of the present invention and a second drug substance.

A combination of the present invention and a second drug substance as a combination partner may be administered by any conventional route, for example as set out above for a compound of the present invention. A second drug may be administered in dosages as appropriate, e.g. in dosage ranges which are similar to those used for single treatment, or, e.g. in case of synergy, even below conventional dosage ranges.

Pharmaceutical compositions according to the present invention may be manufactured according, e.g. analogously, to a method as conventional, e.g. by mixing, granulating, coating, dissolving or lyophilizing processes. Unit dosage forms may contain, for example, from about 0.1 mg to about 1500 mg, such as 1 mg to about 1000 mg.

Pharmaceutical compositions comprising a combination of the present invention and pharmaceutical compositions comprising a second drug as described herein, may be provided as appropriate, e.g. according, e.g. analogously, to a method as conventional, or as described herein for a pharmaceutical composition of the present invention.

By the term “second drug substance” is meant a chemotherapeutic drug, especially any chemotherapeutic agent other than a compound of the present invention, such as a compound of formula I.

For example, a second drug substance as used herein includes e.g. -other compounds, than compounds of the present inventions which interact in cell adhesion, migration and activation activity of LFA-1 with its ligands, e.g. including antibodies and low molecular weight compounds,
anti-inflammatory and/or immunomodulatory drugs,
anti-allergic drugs,
anticancer drugs.

Anti-inflammatory and/or immunomodulatory drugs which are prone to be useful in combination with a compound of the present invention include e.g.
mediators, e.g. inhibitors, of mTOR activity, including rapamycin of formula

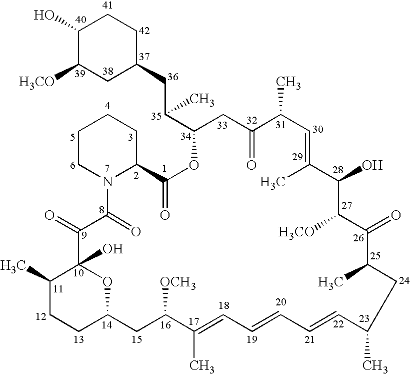

and rapamycin derivatives, e.g. including

40-O-alkyl-rapamycin derivatives, such as 40-O-hydroxyalkyl-rapamycin derivatives, such as 40-O-(2-hydroxy)-ethyl-rapamycin (everolimus),

32-deoxo-rapamycin derivatives and 32-hydroxy-rapamycin derivatives, such as 32-deoxorapamycin,

16-O-substituted rapamycin derivatives such as 16-pent-2-ynyloxy-32-deoxorapamycin,

16-pent-2-ynyloxy-32 (S or R) -dihydro-rapamycin, 16-pent-2-ynyloxy-32(S or R)-dihydro-40-O-(2-hydroxyethyl)-rapamycin,

rapamycin derivatives which are acylated at the oxygen group in position 40, e.g. 40-[3-hydroxy-2-(hydroxy-methyl)-2-methylpropanoate]-rapamycin (also known as CC1779), rapamycin derivatives which are substituted in 40 position by heterocyclyl, e.g. 40epi-(tetrazolyl)-rapamycin (also known as ABT578), the so-called rapalogs, e.g. as disclosed in WO9802441, WO0114387 and WO0364383, such as AP23573, and

compounds disclosed under the name TAFA-93 AP23464, AP23675, AP23841 and biolimus (e.g. biolimus A9).

mediators, e.g. inhibitors, of calcineurin, e.g. cyclosporin A, FK 506;

ascomycins having immuno-suppressive properties, e.g. ABT-281, ASM981;

corticosteroids; cyclophosphamide; azathioprene; leflunomide; mizoribine;

mycophenolic acid or salt; mycophenolate mofetil;

15-deoxyspergualine or an immunosuppressive homologue, analogue or derivative thereof;

mediators, e.g. inhibitors, of bcr-abl tyrosine kinase activity;

mediators, e.g. inhibitors, of c-kit receptor tyrosine kinase activity;

mediators, e.g. inhibitors, of PDGF receptor tyrosine kinase activity, e.g. Gleevec (imatinib);

mediators, e.g. inhibitors, of p38 MAP kinase activity,

mediators, e.g. inhibitors, of VEGF receptor tyrosine kinase activity,

mediators, e.g. inhibitors, of PKC activity, e.g. as disclosed in WO0238561 or WO382859, e.g. the compound of Example 56 or 70;

mediators, e.g. inhibitors, of JAK3 kinase activity, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin4-yl)-amino]-piperdin-1-yl}-3-oxo-propionitrile, in free form or in a pharmaceutically acceptable salt form, e.g. mono-citrate (also called CP-690,550), or a compound as disclosed in WO2004052359 or WO2005066156;

mediators, e.g. agonists or modulators of S1P receptor activity, e.g. FTY720 optionally phosphorylated or an analog thereof, e.g. 2-amino-2-[4-(3-benzyloxyphenylthioy)-2-chlorophenyl]ethyl-1,3-propanediol optionally phosphorylated or 1-{(4-[1-(4-cyclohexyl-3-trifluoromethyl-benzyloxyimino)-ethyl]-2-ethyl-benzyl}-azetidine-3-carboxylic acid or its pharmaceutically acceptable salts;

immunosuppressive monoclonal antibodies, e.g., monoclonal antibodies to leukocyte receptors, e.g., Blys/BAFF receptor, MHC, CD2, CD3, CD4, CD7, CD8, CD25, CD28, CD40, CD45, CD52, CD58, CD80, CD86, IL-12 receptor, IL-17 receptor, IL-23 receptor or their ligands;

other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA41g (for ex. designated ATCC 68629) or a mutant thereof, e.g. LEA29Y;

mediators, e.g. inhibitors of adhesion molecule activities, e.g. LFA-1 antagonists, ICAM-1 or −3 antagonists, VCAM-4 antagonists or VLA-4 antagonists,

mediators, e.g. antagonists of CCR9 activity,

mediators, e.g. inhibitors, of MIF activity,

5-aminosalicylate (5-ASA) agents, such as sulfasalazine, Azulfidine®, Asacol®, Dipentum®, Pentasa®), Rowasa®, Canasa®, Colazal®, e.g. drugs containing mesalamine; e.g mesalazine in combination with heparin;

mediators, e.g. inhibitors, of TNF-alpha activity, e.g. including antibodies which bind to TNF-alpha, e.g. infliximab (Remicade®), thalidomide, lenalidomide,

nitric oxide releasing non-steriodal anti-inlammatory drugs (NSAIDs), e.g. including COX-inhibiting NO-donating drugs (CINOD);

phospordiesterase, e.g. mediatorssuch as inhibitors of PDE4B activity,

mediators, e.g. inhibitors, of caspase activity,

mediators, e.g. agonists, of the G protein coupled receptor GPBAR1,

mediators, e.g. inhibitors, of ceramide kinase activity,

‘multi-functional anti-inflammatory’ drugs (MFAIDs), e.g. cytosolic phoshpholipase A2 (cPLA2) inhibitors, such as membrane-anchored phospholipase A2 inhibitors linked to glycosaminoglycans;

antibiotics, such as penicillins, cephalosporins, erythromycins, tetracyclines, sulfonamides, such as sulfadiazine, sulfisoxazole; sulfones, such as dapsone; pleuromutilins, fluoroquinolones, e.g. metronidazole, quinolones such as ciprofloxacin; levofloxacin; probiotics and commensal bacteria e.g. Lactobacillus, Lactobacillus reuteri;

antiviral drugs, such as ribivirin, vidarabine, acyclovir, ganciclovir, zanamivir, oseltamivir phosphate, famciclovir, atazanavir, amantadine, didanosine, efavirenz, foscarnet, indinavir, lamivudine, nelfinavir, ritonavir, saquinavir, stavudine, valacyclovir, valganciclovir, zidovudine.

Anti-inflammatory which are prone to be useful in combination with a compound of the present invention include e.g. non-steroidal antiinflammatory agents (NSAIDS) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); cyclooxygenase-2 (COX-2) inhibitors such as celecoxib; inhibitors of phosphodiesterase type IV (PDE-IV); antagonists of the chemokine receptors, especially CCR-1, CCR-2, and CCR-3; cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; anticholinergic agents such as muscarinic antagonists (ipratropium bromide); other compounds such as theophylline, sulfasalazine and aminosalicylates, e.g. 5-aminosalicylic acid and prodrugs thereof, antirheumatics.

Antiallergic drugs which are prone to be useful in combination with a compound of the present invention include e.g. antihistamines (H1-histamine antagonists), e.g. bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, praniukast, iralukast, pobilukast, SKB-106,203), leukotriene biosynthesis inhibitors (zileuton, BAY-1005); bronchodilators, antiasthmatics (mast cell stabilizers).

The weight ratio of a compound of the present invention to other drug may vary, e.g. dependent on the activity of the combination partners used, the kind of disease treated, and will further depend upon the effective dose of each ingredient and may be established e.g. by instructions given for the other drug and testing, e.g. according, e.g. analogously, to a method as conventional.

The chemical names of the compounds of the present invention as indicated herein are copied from ISIS, version 2.5 (AutoNom 2000 Name).

In the following Examples all temperatures indicated are in degree Celsius (° C). The following abbraviations are used:

| | |
|---|---|
| EtOAc | ethyl acetate |
| THF | tetrahydrofurane |

### PREPARATION EXAMPLE 1

### N-[7a-(4-Cyano-benzyl)-6-(3,5-dichloro-phenyl)-7-hydroxy-5-oxo-2,3,5,7a-tetrahydro-1H-pyrrolizin-2-yl]-acetamide (compound of TABLE example 1)

A) (2S,4S)4-Azido-pyrrolidine-1,2-dicarboxylic acid 1-allyl ester 2-methyl ester

To a cooled solution of 45 g of (2S,4R)4-hydroxy-pyrrolidine-1,2-dicarboxylic acid 1-allyl ester 2-methyl ester and 65 g of triphenylphosphine in 700 ml of THF are added 46 9 of diethyl azodicarboxylate (DEAD) followed by 53 ml of diphenyl phosphoryl azide (DPPA). The mixture obtained is allowed to warm to rt and solvent is evaporated. The evaporation residue obtained is subjected to chromatography. (2S,4S)-4-Azido-pyrrolidine-1,2-dicarboxylic acid 1-allyl ester 2-methyl ester is obtained in the form of a slightly yellowish oil. MS: 277 (MNa⁺); ¹H-NMR and ¹³C-NMR data are in accordance with the proposed structure.

Ba) (2S,4S)-4-Azido-2-(4-cyano-benzyl)-pyrrolidine-1,2-dicarboxylic acid, 1-allyl ester, 2-methyl ester and
Bb) (2S,4R)4-Azido-2-(4-cyano-benzyl)-pyrrolidine-1,2-dicarboxylic acid 1-allyl ester 2-methyl ester

110 ml of an 1 M-solution of lithium-bis-trimetylsilylamide in THF are added to a cooled (−78°), stirred solution of 21.4 g of (2S,4S)4-azido-pyrrolidine-1,2-dicarboxylic acid 1-allyl ester 2-methyl ester in 200 ml of THF in such a manner that the temperature not exceeds −50°. To the mixture obtained 25.6 g of 4-cyano benzylbromide are added and stirring and cooling is continued for additional 40 minutes. To the mixture obtained 1200 ml of EtOAc and 300 ml of 1N HCl are added, the phases obtained are separated and the organic layer obtained is washed, dried, solvent is evaporated and the evaporation residue is subjected to chromatography. (2S,4S)4-Azido-2-(4-cyano-benzyl)-pyrrolidine-1,2-dicarboxylic acid, 1-allyl ester, 2-methyl ester (14 g) and (2S,4R)4-azido-2-(4-cyano-benzyl)-pyrrolidine-1,2-dicarboxylic acid 1-allyl ester 2-methyl ester (7 g) are obtained, each in the form of a colorless oil. Both compounds: MS: 3921761 (MNa+/2MNa+); ¹H-NMR and ¹³C-NMR data are in accordance with the proposed structure.

C) (2S,4S)-4-Amino-2-(4-cyano-benzyl)-pyrrolidine-1,2-dicarboxvlic acid₁₋₁-allyl ester, 2-methyl ester

To a stirred solution of 3.5 g of (2S,4S)4-azido-2-(4-cyano-benzyl)-pyrrolidine-1,2-dicarboxylic acid 1-allyl ester 2-methyl ester in 150 ml of CH₃CN in 30 ml of CH₃COOH are added 10 g of zinc powder in one portion at rt and the mixture obtained is stirred for. 15 minutes. The mixture obtained is filtered and to the filtrate obtained EtOAc and a saturated aqeous NAHCO₃-solution are added. To the stirred biphasic mixture obtained solid NAHCO₃ is added until no further evolution of CO₂ is determined. The phases obtained are separated, the organic layer obtained is washed and dried and solvent is evaporated (2S,4S)-4-Amino-2-(4-cyano-benzyl)-pyrrolidine-1,2-dicarboxylic acid, 1-allyl ester, 2-methyl ester is obtained in the form of an oil. MS: 344/366/709 (MNH+/MNa+/2MNa+); ¹H-NMR and ¹³C-NMR data are in accordance with the proposed structure.

D) (2S,4S)-4-Acetylamino-2-(4-cyano-benzyl)-pyrrolidine-1 2-dicarboxylic acid 1-allyl ester 2-methyl ester

To a stirred solution of the (2S,4S)4-Amino-2-(4-cyano-benzyl)-pyrrolidine-1,2-dicarboxylic acid 1-allyl ester 2-methyl ester (e.g. obtained according to step C)) in 50 ml of THF are added 7 ml of pyridine followed by 4 ml of acetic acid anhydride at rt. The mixture obtained is stirred for 20 minutes at rt, and 600 ml of EtOAc, 200 ml of a saturated aqeous NAHCO₃-solution and 200 ml of brine are added. Two phases obtained, are separated and the organic layer obtained is washed, dried and solvent is evaporated. The evaporation residue obtained is subjected to chromatography.

(2S,4S)4-Acetylamino-2-(4-cyano-benzyl)-pyrrolidine-1,2-dicarboxylic acid 1-allyl ester 2-methyl ester is obtained in the form of a crystalline solid.

MS: 408/793 (MNa+/2MNa+); ¹H-NMR and ¹³C-NMR data are in accordance with the proposed structure.

E) (2S,4S)4-Acetylamino-2-(4-cyano-benzyl)-pyrrolidine-2-carboxylic acid methyl ester

To a stirred solution of 2.53 g of (2S,4S)-4-acetylamino-2-(4-cyano-benzyl)-pyrrolidine-1,2-dicarboxylic acid 1-allyl ester 2-methyl ester in 150 ml of THF are added 3.88 g of 1,4-diaza-bicyclo[2.2.2]octane (DABCO) and 782 mg of tetrakis-(triphenylphosphine)-palladium at rt. The mixture obtained is stirred for 35 minutes at rt, 200 ml of EtOAc and 200 ml of a saturated aqeous NAHCO₃-solution are added, the phases obtained are separated, the organic layer obtained is washed, dried and solvent is evaporated.

MS: 324 (MNa+), ¹H-NMR and ¹³C-NMR data are in accordance with the proposed structure.

F) (2S,4S)-4-Acetylamino-2-(4-cyano-benzyl)-1-[2-(3,5-dichloro-Phenyl)-acetyl]-pyrrolidine 2-carboxylic acid methyl ester

(2S,4S)4-Acetylamino-2-(4-cyano-benzyl)-pyrrolidine-2-carboxylic acid methyl ester, e.g. obtained according to step D), is treated with 100 ml of CH₃CN and 40 ml of water and to the mixture obtained 4 g of KHCO₃ and 2.6 g of 2,5-dichlorophenyl acetic acid are added in one portion. The mixture obtained is stirred for 35 minutes at rt, 300 ml of EtOAc, 50 ml of brine and 50 ml of H₂O are added and the phases obtained are separated. The organic layer obtained is washed, dried and solvent is evaporated. The evaporation residue obtained is subjected to chromatography. (2S,4S)4-Acetylamino-2-(4-cyano-benzyl)-1-[2-(3,5-dichloro-phenyl)-acetyl]-pyrrolidine 2-carboxylic acid methyl ester is obtained in the form of an amorphous powder.

MS: 510 (MNa+), ¹H-NMR and ¹³C-NMR data are in accordance with the proposed structure.

G) N-[(2S.7aS)-7a-(4-Cyano-benzyl)-6-(3,5-dichloro-phenyl)-7-hydroxy-5-oxo-2,3,5,7a-tetrahydro-1H-pyrrolizin-2-yl]-acetamide

1.75 g of potassium hexamethyl silazane (KHMDS).are added to a stirred solution of 3.03 g of (2S,4S)4-acetylamino-2-(4-cyano-benzyl)-1-[2-(3,5-dichloro-phenyl)-acetyl]-pyrrolidine 2-carboxylic acid methyl ester in 150 ml of dry THF and the mixture obtained is stirred for some minutes. To the mixture obtained 1N HCl and EtOAc are added. The phases obtained are separated, the organic layer obtained is washed, dried and solvent is evaporated. N-[(2S,7aS)-7a-(4-Cyano-benzyl)-6-(3,5-dichloro-phenyl)-7-hydroxy-5-oxo-2,3,5,7a-tetrahydro-1H-pyrrolizin-2-yl]-acetamide is obtained.

MS: 478 (MNa+), ¹H-NMR and ¹³C-NMR data are in accordance with the proposed structure.

### PREPARATION EXAMPLES 2 AND 3

### N-[7a-(4-Cyano-benzyl)-6-3,5-dichloro-phenyl)-7-methoxy-5-oxo-2,3,5,7a-tetrahydro-1H-pyrrolizin-2-yl]-acetamide (Preparation Example 2, compound of TABLE Example 2) and

### N-[7a-(4-Cyano-benzyl)-6-(3,5-dichloro-phenyl)-5-methoxy-7-oxo-2,3,7,7a-tetrahydro-1H-pyrrolizin-2-yl]-acetamide (Preparation Example 3, compound of TABLE Example 3)

To 1.8 g of -[(2S,7aS)-7a-(4-Cyano-benzyl)-6-(3,5-dichloro-phenyl)-7-hydroxy-5-oxo-2,3,5,7a-tetrahydro-1H-pyrrolizin-2-yl]-acetamide in 60 ml of CH₂Cl₂ is added a solution of CH₂N₂ in diethylether until the characteristic yellow color of unreacted CH₂N₂ disappears. Unreacted CH₂N₂ is removed with a stream of argon and solvent is evaporated. The evaporation residue obtained is subjected to chromatography.

N-[(2S,7aS)-7a-(4-Cyano-benzyl)-6-(3,5-dichloro-phenyl)-7-methoxy-5-oxo-2,3,5,7a-tetrahydro-1H-pyrrolizin-2-yl]-acetamide (Example 2) and N-[(2S,7aS)-7a-(4-Cyano-benzyl)-6-(3,5-dichloro-phenyl)-5-methoxy-7-oxo-2,3,7,7a-tetrahydro-1H-pyrrolizin-2-yl]-acetamide (Example 3) are obtained.

Both compounds: MS: 368/713 (MNa+/2MNa+), ¹H-NMR and ¹³C-NMR data are in accordance with the proposed structures.

### PREPARATION EXAMPLE 4

### 4-[6-(tert-Butyl-dimethyl-silanyloxy)-3-oxo-2-phenyl-5,6,7,7a-tetrahydro-3H-pyrrolizin-1-yloxymethyl]-benzonitrile, (compound of TABLE Example 14),

A compound of formula

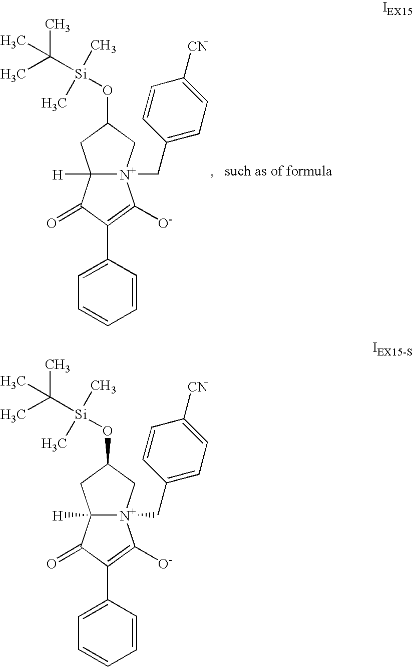

(compound of TABLE Example 15), and
4-[6-(tert-Butyl-dimethyl-silanyloxy)-1,3-dioxo-2-phenyl-hexahydro-pyrrolizin-2-ylmethyl]-benzonitrile, (compound of TABLE Example 16)

A mixture of 840 mg of (6R,7aR)-6-(tert-butyl-dimethyl-silanyloxy)-1-hydroxy-2-phenyl-5,6,7,7a-tetrahydro-pyrrolizin-3-one, 2 ml of N-ethyl-diisopropyl amine (Hünig base) and 1.09 g of 4-cyano-benzylbromide in 50 ml of CH₂Cl₂ are stirred for 15 hours at rt, the mixture obtained is treated with citrate puffer (pH 3) and EtOAc and the phases are separated. The organic layer obtained is washed, dried and solvent is evaporated. The evaopration residue obtained is s subjected to chromatography (silica gel; gradient toluene:acetonitrile=10:1 to 1:1).

322 mg of a compound of formula I_{EX 15-S}, 55 mg of 4-[(6R,7aR)-6-(tert-butyl-dimethyl-silanyloxy)-3-oxo-2-phenyl-5,6,7,7a-tetrahydro-3H-pyrrolizin-1-yloxymethyl]-benzonitrile and 678 mg of 4-[(6S,7aS)-6-(tert-butyl-dimethyl-silanyloxy)-1,3-dioxo-2-phenyl-hexahydro-pyrrolizin-2-ylmethyl]-benzonitrile are obtained.

### PREPARATION EXAMPLE 5

Compound of formula

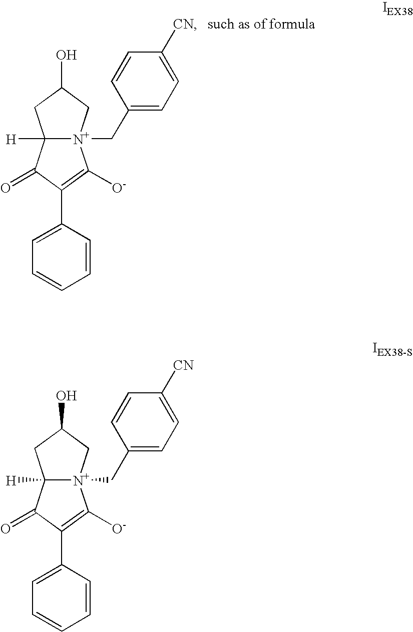

A solution of 255 mg of a compound of formula I_{EX15} in 7 ml of THF and 2.8 ml of 1M tetrabutyl ammonium fluoride in THF is stirred for 20 min at rt. The mixture obtained is treated with 1 N-HCl and EtOAc, the phases obtained are separated and the organic layer obtained is washed and dried. From the mixture obtained solvent is evaporated and the evaporation residue is subjected to chromatography. A compound of formula I_{EX38S} is obtained in the form of a colorless solid. MS: 347/369/715 (MH+/MNa+/2MNa+), ¹H-NMR and ¹³C-NMR data are in accordance with the proposed structure.

### PREPARATION EXAMPLE 6

A compound of formula

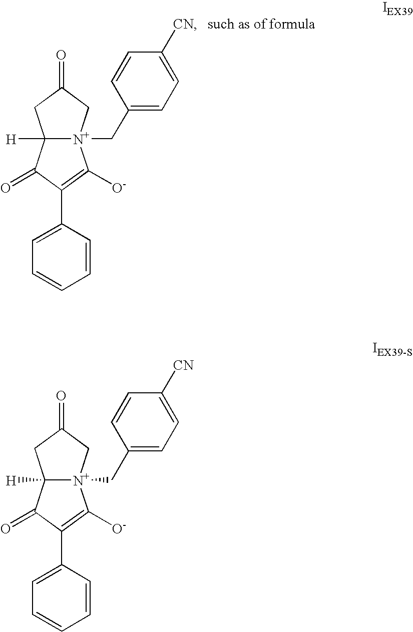

A mixture of 325 mg of 1-hydroxy-1,2-benziodoxol-3(1H)-one 1-oxide and 90 mg of a compound of formula I_{EX38-S} in 5 ml of EtOAc is allowed to react under microwave conditions (100° C.; 65 minutes; Personal chemistry Emrys optimizer). The mixture obtained is s filtrated (removal of excess reagent), concentrated at reduced pressure and subjected to chromatography. A compound of formula I_{EX39-S} is obtained in the form of a solid.

MS: 399/755 (MNa⁺+MeOH/2MNa⁺+2MeOH), ¹H-NMR and ¹³C-NMR data are in accordance with the proposed structure.

### PREPARATION EXAMPLE 7

4-{6-(3,5-Dichloro-phenyl)-2-[5-iodo-4-(3-methyl-butyl)-[1,2,3]triazol-1-yl]-7-methoxy-5-oxo-2,3-dihydro-1H,5H-pyrrolizin-7a-ylmethyl}-benzonitrile (compound of TABLE Example 57),
4-{6-(3,5-Dichloro-phenyl)-7-methoxy-2-[4-(3-methyl-butyryl)-[1,2,3]triazol-1-yl]-5-oxo-2,3-dihydro-1H,5H-pyrrolizin-7a-ylmethyl}-benzonitrile (compound of TABLE Example 58);
4-{643,5-Dichloro-phenyl)-7-methoxy-2-[4-(3-methyl-butyl)-[1,2,3]triazol-1-yl]-5-oxo-2,3-dihydro-1H,5H-pyrrolizin-7a-ylmethy}-benzonitrile, (compound of TABLE Example 59), and
4-{(2S,7aS)-6-(3,5-Dichloro-phenyl)-2-[5-iodo-4-(3-methyl-butyryl)-[1,2,3]triazol-1-yl]-7-methoxy-5-oxo-2,3-dihydro-1H,5H-pyrrolizin-7a-ylmethyl}-benzonitrile (compound of TABLE Example 60)

To a stirred solution of 100 mg of 4-[(2S,7aS)-2-azido-6-(3,5-dichloro-phenyl)-7-methoxy-5-oxo-2,3-dihydro-1H,5H-pyrrolizin-7a-ylmethyl]-benzonitrile (a compound of TABLE example 74) and 0.14 ml of 5-methyl-1-hexyne are added 42.8mg of copper(I)iodide and the mixture obtained is stirred at rt for ca. 20 hours. For working up the mixture obtained is treated with EtOAc and saturated aqueous NaHCO₃ solution. The organic layer obtained is washed with brine, dried and solvent is evaporated. The evaporation residue is subjected to column chromatography.

4-{(2S,7aS)-6-(3,5-Dichloro-phenyl)-2-[5-iodo-4-(3-methyl-butyl)-[1,2,3]triazol-1-yl]-7-methoxy-5-oxo-2,3-dihydro-1H,5H-pyrrolizin-7a-ylmethyl}-benzonitrile; (MS: 698 (MNa+)),

4-{(2S,7aS)-6-(3,5-Dichloro-phenyl)-7-methoxy-2-[4-(3-methyl-butyryl)-[1,2,3]triazol-1-yl]-5-oxo-2,3-dihydro-1H,5H-pyrrolizin-7a-ylmethyl}-benzonitrile (MS: 586 (MNa+)),

4-{(2S,7aS)-6-(3,5-Dichloro-phenyl)-7-methoxy-2-[4-(3-methyl-butyl)-[1,2,3]triazol-1-yl]-5-oxo-2,3-dihydro-1H,5H-pyrrolizin-7a-ylmethyl}-benzonitrile; (MS: 572 (MNa+)), and

4-{(2S,7aS)-6-(3,5-Dichloro-phenyl)-2-[5-iodo-4-(3-methyl-butyryl)-[1,2,3]triazol-1-yl]-7-methoxy-5-oxo-2,3-dihydro-1H,5H-pyrrolizin-7a-ylmethyl}-benzonitrile (MS: 712 (MNa+)) are obtained in the form of solids.

Analogously to a method as described in previous examples, but using appropriate starting materials (intermediates), compounds of formula I, which are compounds of formula

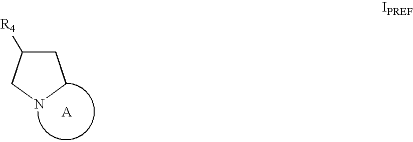

wherein ring A is a group of formula A1, A2, A3, A4 or A5 and wherein R₁, R₂, R₃, R₄, R₅ and R₆ are as defined in TABLE 1 below, are obtained. Analytical data (mass spectroscopy) is also set out in TABLE 1. ¹H-NMR and ¹³C-NMR data of the compounds set out in TABLE 1 below have been found to be in accordance with the corresponding proposed structure. Examples 33, 34, 38, 39, 44 and 45 refer to compounds which are useful as intermediates for the preparation of compounds of formula I_{PREF}.

TABLE 1

| | Example 1 | Example 2 | Example 3 | Example 4 | Example 5 | Example 6 |
|---|---|---|---|---|---|---|
| Ring A | Group A5 | Group A5 | Group A3 | Group A3 | Group A3 | Group A3 |
| R₁ | — | — | — | — | — | — |
| R₂ | 3,5-dichlorophenyl | 3,5-dichlorophenyl | 3,5-dichlorophenyl | 3,5-dichlorophenyl | 3,5-dichlorophenyl | 3,5-dichlorophenyl |
| R₃ | 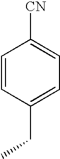 | 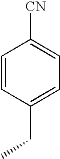 | 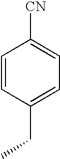 | 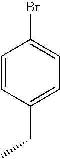 | 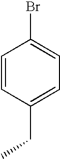 | 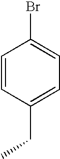 |
| R₄ | 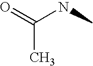 | 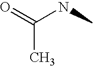 | 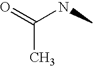 | 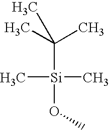 | 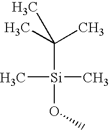 | 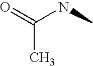 |
| R₅ | — | — | — | — | — | — |
| R₆ | OH | OCH₃ | OCH₃ | OCH₃ | methoxybutoxy | OCH₃ |
| DATA | MS: 478 (MNa⁺) | MS: 368/713 | MS: 368/713 | MS: 596 (MH⁺) | MS: 668 (MH⁺) | MS: 545/1067 |
| | | (MNa⁺/2MNa⁺) | (MNa⁺/2MNa⁺) | | | (MNa⁺/2MNa⁺) |

| | Example 7 | Example 8 | Example 9 | Example 10 | Example 11 | Example 12 |
|---|---|---|---|---|---|---|
| Ring A | Group A3 | Group A3 | Group A3 | Group A5 | Group A5 | Group A5 |
| R₁ | — | — | — | — | — | — |
| R₂ | 3,5-dichlorophenyl | phenyl | phenyl | phenyl | phenyl | phenyl |
| R₃ | 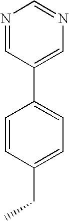 | 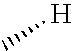 | 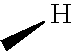 | 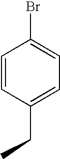 | 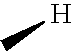 | 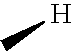 |
| R₄ | 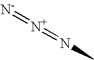 | 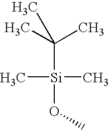 | 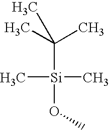 | 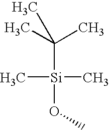 | 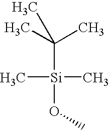 | 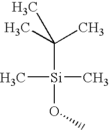 |
| R₅ | — | — | — | — | — | — |
| R₆ | OCH₃ | OCH₃ | OCH₃ | OH | OCH₃ | OH |
| DATA | MS: 529/1035 | MS: 382/741 | MS: 382/741 | MS: 536/1049 | MS: 382/741 | MS: 346/368/713 |
| | (MNa⁺/2MNa⁺) | (MNa⁺/2MNa⁺) | (MNa⁺/2MNa⁺) | (MNa⁺/2MNa⁺) | (MNa⁺/2MNa⁺) | (MH⁺/MNa⁺/2MNa⁺) |

| | Example 13 | Example 14 | Example 15 | Example 16 | Example 17 | Example 18 |
|---|---|---|---|---|---|---|
| Ring A | Group A5 | Group A5 | Group A1 | Group A4 | Group A5 | Group A5 |
| R₁ | — | — | 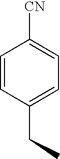 | — | — | — |
| R₂ | phenyl | phenyl | phenyl | phenyl | 3,5-dichlorophenyl | 3,5-dichlorophenyl |
| R₃ | 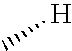 | 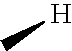 | 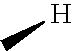 | 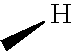 | 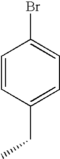 | 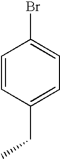 |
| R₄ | 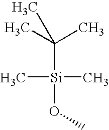 | 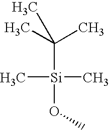 | 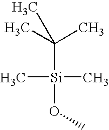 | 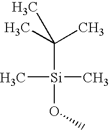 | 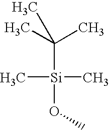 | 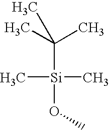 |
| R₅ | — | — | — | 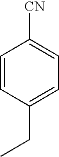 | — | — |
| R₆ | OH | 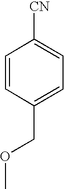 | — | — | OH | OCH₃ |
| DATA | MS: 346/368/713 | MS: 461/483/943 | MS: 461/483/943 | MS: 461/483/943 | MS: 582 (MH⁺) | MS: 596 (MH⁺) |
| | (MH⁺/MNa⁺/2MNa⁺) | (MH⁺/MNa⁺/2MNa⁺) | (MH⁺/MNa⁺/2MNa⁺) | (MH⁺/MNa⁺/2MNa⁺) | | |

| | Example 19 | Example 20 | Example 21 | Example 22 | Example 23 | Example 24 |
|---|---|---|---|---|---|---|
| Ring A | Group A5 | Group A5 | Group A5 | Group A5 | Group A5 | Group A5 |
| R₁ | — | — | — | — | — | — |
| R₂ | 3,5-dichlorophenyl | phenyl | phenyl | phenyl | phenyl | phenyl |
| R₃ | 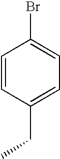 | 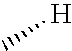 | 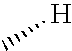 | 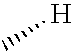 | 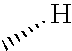 | 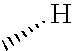 |
| R₄ | 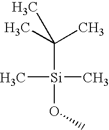 | 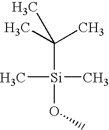 | 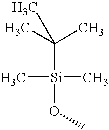 | 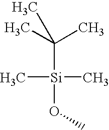 | 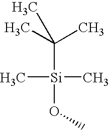 | 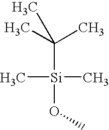 |
| R₅ | — | — | — | — | — | — |
| R₆ | methoxybutoxy | OCH₃ | 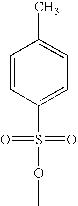 | 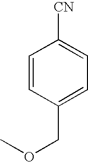 | 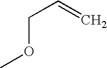 | 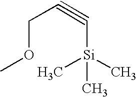 |
| DATA | MS: 668 (MH⁺) | MS: 382/741 | MS: 522/1021 | MS: 483/943 | MS: 408/793 | MS: 478/933 |
| | | (MNa⁺/2MNa⁺) | (MNa⁺/2MNa⁺) | (MNa⁺/2MNa⁺) | (MNa⁺/2MNa⁺) | (MNa⁺/2MNa⁺) |

| | Example 25 | Example 26 | Example 27 | Example 28 | Example 29 | Example 30 |
|---|---|---|---|---|---|---|
| Ring A | Group A5 | Group A5 | Group A5 | Group A5 | Group A5 | Group A5 |
| R₁ | — | — | — | — | — | — |
| R₂ | 3,5-dichlorophenyl | 3,5-dichlorophenyl | 3,5-dichlorophenyl | 3,5-dichlorophenyl | 3,5-dichlorophenyl | 3,5-dichlorophenyl |
| R₃ | 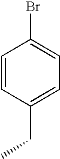 | 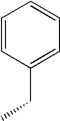 | 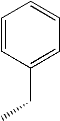 | 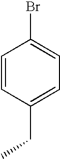 | 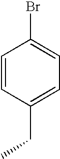 | 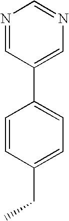 |
| R₄ | 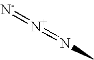 | 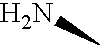 | 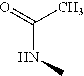 | 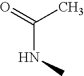 | 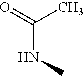 | 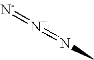 |
| R₅ | — | — | — | — | — | — |
| R₆ | OH | OH | 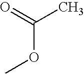 | OH | OCH₃ | OCH₃ |
| DATA | MS: 492 (MH⁺) | MS: 389 (MH⁺) | MS: 495 (MNa⁺) | MS: 530 (MH⁺) | MS: 545/1067 | MS: 529/1035 |
| | | | | | (MNa₊/2MNa₊) | (MNa⁺/2MNa⁺) |

| | | | Example 33 | Example 34 | | | |
|---|---|---|---|---|---|---|---|
| | Example 31 | Example 32 | Intermediate | Intermediate | Example 35 | Example 36 | Example 37 |
| Ring A | Group A5 | Group A5 | Group A5 | Group A5 | Group A1 | Group A1 | Group A1 |
| R₁ | — | — | — | — | 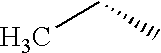 | 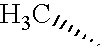 | 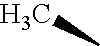 |
| R₂ | 3,5-dichlorophenyl | 3,5-dichlorophenyl | phenyl | 3,5-dichlorophenyl | phenyl | phenyl | phenyl |
| R₃ | 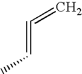 | 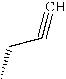 | 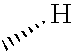 | 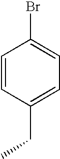 | 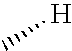 | 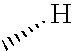 | 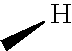 |
| R₄ | 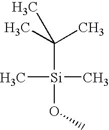 | 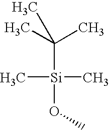 | 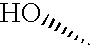 | 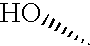 | 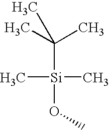 | 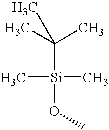 | 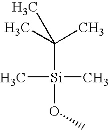 |
| R₅ | — | — | — | — | — | | — |
| R₆ | OH | OH | OH | OH | — | — | — |
| DATA | MS: 474/925 | MS: 474/925 | MS: 254/485 | MS: 579 (MNa⁺ + | MS: 396/769 | MS: 382/741 | MS: 382/741 |
| | (MNa⁺/2MNa⁺) | (MNa⁺/2MNa⁺) | (MNa⁺/2MNa⁺) | EtOAc) | (MNa⁺/2MNa⁺) | (MNa⁺/2MNa⁺) | (MNa⁺/2MNa⁺) |

| | Example 38 | Example 39 | | | | | Example 44 |
|---|---|---|---|---|---|---|---|
| | Intermediate | Intermediate | Example 40 | Example 41 | Example 42 | Example 43 | Intermediate |
| Ring A | Group A1 | Group A1 | Group A1 | Group A1 | Group A1 | Group A1 | Group A1 |
| R₁ | 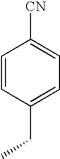 | 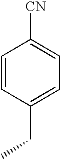 | 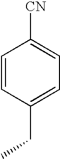 | 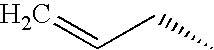 | 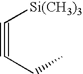 | 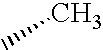 | 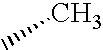 |
| R₂ | phenyl | phenyl | phenyl | phenyl | phenyl | phenyl | phenyl |
| R₃ | 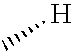 | 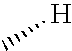 | 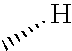 | 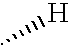 | 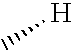 | 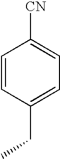 | 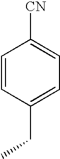 |
| R₄ | 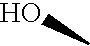 | 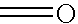 | 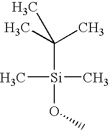 | 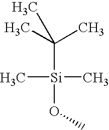 | 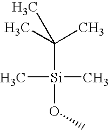 | 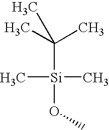 | 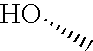 |
| R₅ | — | — | — | — | — | — | — |
| R₆ | — | — | — | — | — | — | — |
| DATA | MS: 347/369/715 | MS: 399/755 | MS: 483/943 | MS: 408/793 | MS: 478/933 | MS: 497/971 | MS: 383/743 |
| | (MH+/MNa+/ | (MNa⁺ + MeOH/ | (MNa⁺/2MNa⁺) | (MNa⁺/2MNa⁺) | (MNa⁺/2MNa⁺) | (MNa⁺/2MNa⁺) | (MNa⁺/2MNa⁺) |
| | 2MNa+) | 2MNa⁺ + 2MeOH) | | | | | |

| | Example 45 | | | | | |
|---|---|---|---|---|---|---|
| | Intermediate | Example 46 | Example 47 | Example 48 | Example 49 | Example 50 |
| Ring A | Group A5 | Group A5 | Group A5 | Group A5 | Group A5 | Group A5 |
| R₁ | — | — | — | — | — | |
| R₂ | 3,5-dichlorophenyl | 3,5-dichlorophenyl | 3,5-dichlorophenyl | 3,5-dichlorophenyl | 3,5-dichlorophenyl | 3,5-dichlorophenyl |
| R₃ | 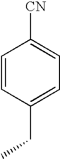 | 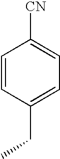 | 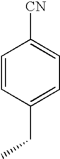 | 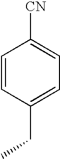 | 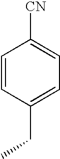 | 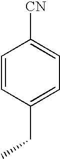 |
| R₄ | 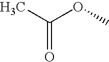 | 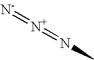 | 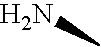 | 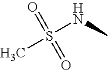 | 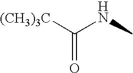 | 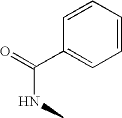 |
| R₅ | — | — | — | — | — | — |
| R₆ | OCH₃ | OH | OCH₃ | OCH₃ | OCH₃ | OCH₃ |
| DATA | MS: 493/963 | MS: 462 (MNa+) | MS: 428 (MH+), | MS: 528 (MNa+) | MS: 534 (MNa+) | MS: 5554 (MNa+) |
| | (MNa⁺/2MNa⁺) | | 450 (MNa+) | | | |

| | Example 51 | Example 52 |
|---|---|---|
| Ring A | Group A5 | Group A5 |
| R₁ | | |
| R₂ | 3,5-dichlorophenyl | 3,5-dichlorophenyl |
| R₃ | 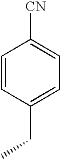 | 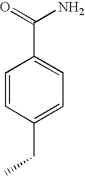 |
| R₄ | 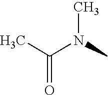 | 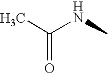 |
| R₅ | — | — |
| R₆ | OCH₃ | OCH₃ |
| DATA | MS: 506/989 | MS: 510/997 |
| | (MNa⁺/2MNa⁺) | (MNa⁺/2MNa⁺) |

Analogously to a method as described in previous examples, but using appropriate starting materials (intermediates), compounds of formula

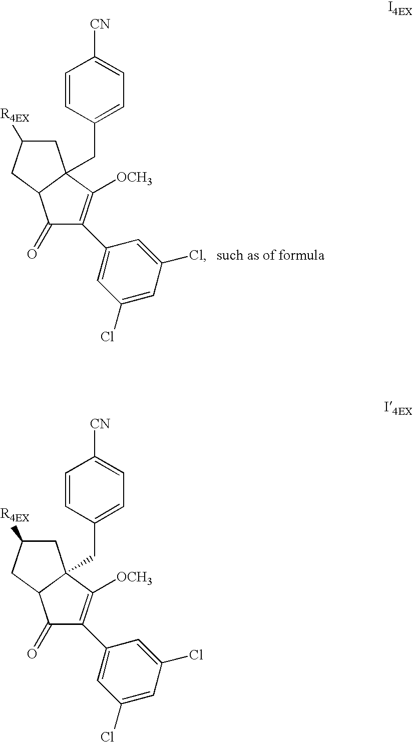

wherein R_{4EX} is as defined in TABLE 2 below are obtained. Analytical data (mass spectroscopy) is also set out in TABLE 2. ¹H-NMR and ¹³C-NMR data of the compounds as defined in TABLE 2 below have been found to be in accordance with the corresponding proposed structure.

TABLE 2

| | Example 53 | Example 54 | Example 55 | Example 56 | Example 57 | Example 58 |
|---|---|---|---|---|---|---|
| R₄ | 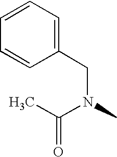 | 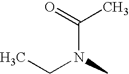 | 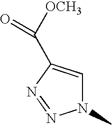 | 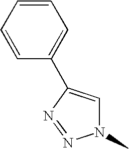 | 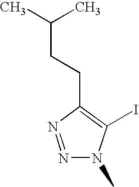 | 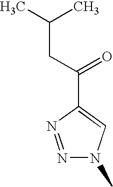 |
| DATA | MS: 582/1141 | MS: 582/1141 | MS: 560 (MNa+) | MS: 578 (MNa+) | MS: 698 (MNa+) | MS: 586 (MNa+) |
| | (MNa⁺/2MNa⁺) | (MNa⁺/2MNa⁺) | | | | |

| | Example 59 | Example 60 | Example 61 | Example 62 | Example 63 | Example 64 | Example 65 |
|---|---|---|---|---|---|---|---|
| R₄ | 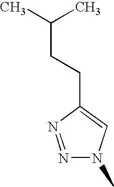 | 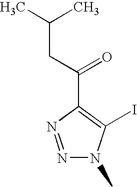 | 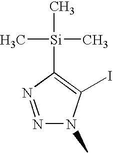 | 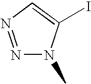 | 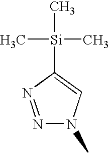 | 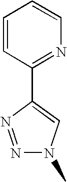 | 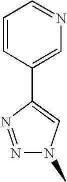 |
| DATA | MS: 572 (MNa+) | MS: 712 (MNa+) | MS. 700 (MNa+) | MS: 628 (MNa+) | MS: 574 (MNa+) | MS: 579 (MNa+) | MS: 579 (MNa+) |

| | Example 66 | Example 67 | Example 68 | Example 69 | Example 70 | Example 71 | Example 72 |
|---|---|---|---|---|---|---|---|
| R₄ | 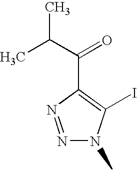 | 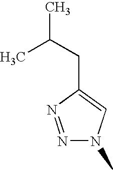 | 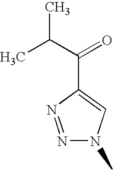 | 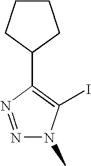 | 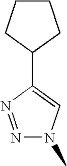 | 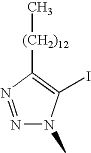 | 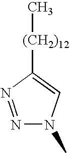 |
| DATA | MS: 698 (MNa+) | MS: 558 (MNa+) | MS: 572 (MNa+) | 696 (MNa+) | 570 (MNa+) | 810 (MNa+) | MS: 684 (MNa+) |

| | Example 73 | Example 74 |
|---|---|---|
| R₄ | 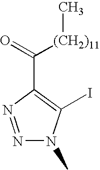 | 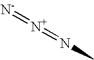 |
| DATA | MS: 824 (MNa+) | MS: 476 (MNa+), |

1. A compound of formula 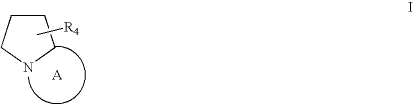
wherein
ring A is a group of formula 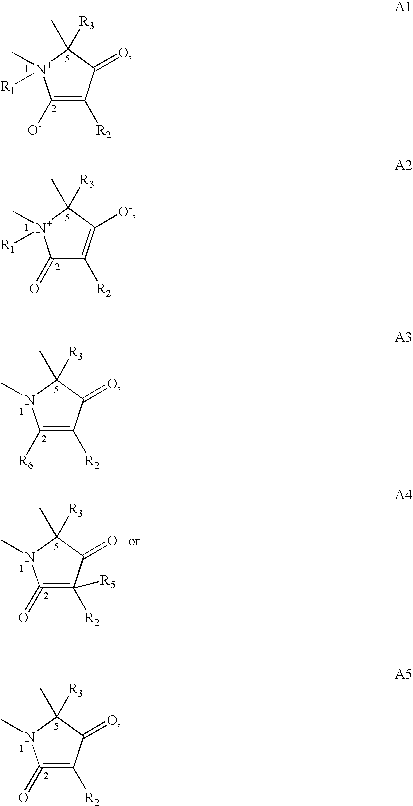
R₁ is
(C₁₋₁₈)alkyl, (C₂₋₁₈)alkenyl, (C₂₋₁₈)alkynyl, or
(C₁₋₁₆)alkyl, (C₂₋₁₈)alkenyl, (C₂₋₁₈)alkynyl, substituted by
(C₁₋₄)alkoxy,
a silyl or silyloxy group,
(C₃₋₁₈)cycloalkyl,
(C₆₋₁₈)aryl, or
heterocyclyl,
R₂ is (C₃₋₁₈)cycloalkyl, (C₃₋₁₈)aryl, or heterocyclyl,
R₃ is hydrogen or unsubstituted (C₁₋₈)alkyl, (C₂₋₄)alkenyl, (C₂₋₈)alkynyl, or (C₁₋₈)alkyl, (C₂₋₈)alkenyl, (C₂₋₈)alkynyl, substituted by
(C₃₋₁₈)cycloalkyl,
(C₃₋₁₈)aryl, or
heterocyclyl,
R₄ is
tri(C₁₋₆)alkylsilyloxy, N₃, amino, (C₁₋₈)alkylamino, (C₁₋₈)dialkylamino, (C₃₋₈)cycloalkylamino, (C₂₋₁₈)acylamino, ((C₂₋₁₈)acyl)-((C₁₋₄)alkyl))-amino, (C₁₋₄)alkysulfonylamino. (C₆₋₁₂)arylsulfonylamino, (C₃₋₈)cyclohexylsulfonylamino, or R₄ is heterocyclyl comprising at least one nitrogen atom as a heteroatom and being bound via that nitrogen atom to a compound of formula I,
R₅ is hydrogen, unsubstituted (C₁₋₁₈)alkyl, (C₂₋₁₈)alkenyl, (C₂₋₁₈)alkynyl, or (C₁₋₁₈)alkyl, (C₂₋₁₈)alkenyl, (C₂₋₁₈)alkynyl substituted by
(C₁₋₄)alkoxy,
tri(C₁₋₆)alkylsilyl, tri(C₁₋₆)alkylsilyloxy,
(C₃₋₁₈)cycloalkyl,
(C₆₋₁₈)aryl, such as (C₆₋₁₂)aryl, or
heterocyclyl,
R₆ is
OR₇ or SR₇,
R₇ is hydrogen, a group (SO)₂—R₉, wherein R₉ is (C₁₋₄)alkyl or (C₆₋₁₂)aryl, COR₈, CSR₈, unsubstituted (C₁₋₁₈)alkyl, (C₂₋₁₈)alkenyl or (C₂₋₁₈)alkynyl, or (C₁₋₁₈)alkyl, (C₂₋₁₈)alkenyl, or (C₂₋₁₈)alkynyl, substituted by
(C₁₋₄)alkoxy, tri(C₁₋₆)alkylsilyl, tri(C₁₋₆)alkylsilyloxy, (C₃₋₁₈)cycloalkyl, (C₆₋₁₈)aryl, or heterocyclyl,
R₈ is (C₃₋₁₈)cycloalkyl, (C₆₋₁₈)aryl, heterocyclyl, unsubstituted (C₁₋₁₈)alkyl, (C₂₋₁₈)alkenyl or (C₂₋₁₈)alkynyl , or (C₁₋₁₈)alkyl, (C₂₋₁₈)alkenyl or (C₂₋₁₈)alkynyl substituted by
(C₃₋₁₈)cycloalkyl, (C₆₋₁₈)aryl, or heterocyclyl,
wherein
cycloalkyl, aryl or heterocyclyl are unsubstituted or substituted by one or more (C₁₋₁₆)alkyl, (C₂₋₁₆)alkenyl, (C₂₋₁₆)alkynyl, (C₃₋₈)cycloalkyl, phenyl, benzyl, heterocyclyl, (C₁₋₄)haloalkyl, (C₁₋₈)alkoxy, phenoxy, oxo, (C₂₋₁₃)acyl, (C₂₋₁₃)acyloxy, amino, (C₁₋₆)alkylamino, (C₁₋₆)dialkylamino, (C₂₋₁₃)acylamino, nitro, cyano, halogen, (C₁₋₄)alkylsulfonyl, tolylsulfonyl, tri(C₁₋₆) alkylsilyl or tri(C₁ 6)silyloxy, and
wherein heterocyclyl comprises
aliphatic and aromatic heterocyclyl,
3 to 8 ring members,
1 to 4 heteroatoms selected from N, O, S,
fused heterocyclyl, such as heterocyclyl fused with another ring (system), with the proviso that, if R₄ is heterocyclyl, said heterocyclyl comprising at least one nitrogen atom as a heteroatom and being bound via that nitrogen atom to a compound of formula I.

2. A compound according to claim 1, wherein
ring A is as defined in claim 1,
R₁ is unsubstituted (C₁₋₆)alkyl, (C₂₋₆)alkenyl, (C₂₋₆)alkynyl, or (C₁₋₆)alkyl, (C₂₋₆)alkenyl, or (C₂₋₄)alkynyl substituted by
(C₆₋₁₈)aryl,
tri(C₁₋₆)alkylsilyl, tri(C₁₋₆)alkylsilyloxy,
R₂ is optionally substituted (C₆₋₁₈)aryl,
R₃ is hydrogen, (C₂₋₆)alkenyl, (C₂₋₆)alkynyl, or (C₆₋₁₂)aryl(C₁₋₄)alkyl,
R₄ is
tri(C₁₋₆)alkylsilyloxy, N₃, amino, (C₁₋₄)alkylcarbonylamino, (C₁₋₄)alkylsulfonylamino, (C₆₋₁₂)arylcarbonylamino, N-((C₁₋₄)alkylcarbonyl)-N-((C₁₋₄)alkyl)-amino, N-((C₆₋₁₂)aryl(C₁₋₄)alkylcarbonyl)-N-((C₁₋₄)alkyl)-amino, or aromatic heterocyclyl comprising 5 or 6 ring members and at least one nitrogen atom and being bound via that nitrogen atom to a compound of formula I,
R₅ is hydrogen or (C₁₋₄)alkyl substituted by (C₆₋₁₈)aryl,
R₆ is OR₇.
R₇ is hydrogen, (C₁₋₄)alkyl, (C₁₋₆)alkoxy(C₁₋₈)alkyl, (C₂₋₈)alkenyl, (C₂₋₈)alkynyl, (C₂₋₈)alkynyl substituted by tri(C₁₋₄)alkyl, (C₁₋₄)alkyl substituted by phenyl, a group (SO)₂—R₉, or COR₈,
R₈ is (C₁₋₄)alkyl; and
R₉ is (C₆₋₁₂)aryl,
wherein aryl is optionally substituted by (C₁₋₁₆)alkyl, (C₃₋₈)cyclohexyl, (C₁₋₄)alkylcarbonyl, such as methylcarbonyl, (C₁₋₄)alkoxycarbonyl, halogen, cyano, aminocarbonyl, heterocyclyl comprising 5 to 6 ring members and 1 to 4 heteroatoms selected from N, O, S, or tri(C₁₋₆)alkylsilyl.

3. A compound according to claim 1, wherein
R₁ is methyl, ethyl, propen-3yl, 1-(trimethylsilyl)propyn-3yl, or cyaonphenylmethyl,
R₂ is dihalophenyl,
R₃ is hydrogen, propadienyl, propynyl, benzyl, cyanophenylmethyl, halophenylmethyl or pyrimidinophenylmethyl,
R₄ is (tert-butyl)(dimethyl)silyloxy, N₃, amino, methylcarbonylamino, tert-butylcarbonylamino, phenylcarbonylamino, N-methylcarbonyl-N-methyl-amino, N-benzyl-N-methylcarbonyl-amino, N-ethyl-N-methylcarbonyl-amino, methylsulfonylamino, or substituted triazolyl bound to a compound of formula I via a nitrogen heteroatom, wherein triazolyl is substituted by isobutyl, isopentyl, n-tridecanyl, cyclopentyl, phenyl, methyloxycarbonyl, 1-methyl-propylcarbonyl, isorpoylcarbonyl, dodecanylcarbonyl, oxo, halogen, tri(C₁₋₄)alkylylsilyl, or pyridinyl,
R₅ is cyanophenyl,
R₆ is OR₇, and
R₇ is hydrogen, methyl, propenyl, trimethylsilyl-propynyl, cyanophenylmethyl, tolylsulfonyl or methylcarbonyl.

4. A compound of claim 1 in the form of a salt.

5. (canceled)

6. A pharmaceutical composition comprising a compound of claim 1 in association with at least one pharmaceutical excipient.

7. A method of treating disorders mediated by interactions of LFA-1 with its ligands involved in cell adhesion, migration and activation activity, which treatment comprises administering to a subject in need of such treatment a therapeutically effective amount of a compound of claim 1.

8. (canceled)

9. A combination of a compound of claim 1 with at least one second drug substance.

10-11. (canceled)

12. A compound of claim 1 selected from 1,6-Dihydroxy-7a-methyl-2-phenyl-5,6,7,7a-tetrahydro-pyrrolizin-3-one, 7a-(4-Bromo-benzyl)-2-(3,5-dichloro-phenyl)-1,6-dihydroxy-5,6,7,7a-tetrahydro-pyrrolizin-3-one, acetic acid 7a-(4-cyano-benzyl)-6-(3,5-dichloro-phenyl)-7-methoxy-5-oxo-2,3,5,7a-tetrahydro-1H-pyrrolizin-2-yl ester or a compound selected from the group consisting of:

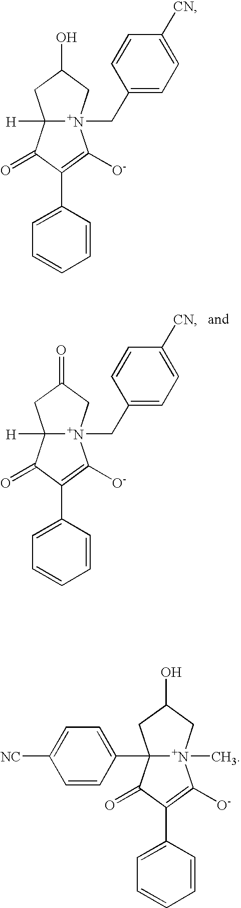

13. A compound of claim 1 selected from (6R,7aS)-1,6-Dihydroxy-7a-methyl-2-phenyl-5,6,7,7a-tetrahydro-pyrrolizin-3-one, or acetic acid (S)-7a-(4-cyano-benzyl)-6-(3,5-dichloro-phenyl)-7-methoxy-5-oxo-2,3,5,7a-tetrahydro-1H-pyrrolizin-2-yl ester or a compound selected from the group consisting of: [chemical structure]
